(19) United States
(12) Patent Application Publication
(10) Pub. No.: US 2025/0084188 A1
(43) Pub. Date: Mar. 13, 2025

(54) ANTI-CHITINASE-3-LIKE PROTEIN 1 ANTIBODY

(71) Applicants: Chungbuk National University Industry-Academic Cooperation Foundation, Cheongju-si (KR); Osong Medical Innovation Foundation, Heungdeok-gu (KR); HWATABIO Co., Ltd, Seo-gu (KR)

(72) Inventors: Jin Tae HONG, Osong-eup (KR); Joo Eon LEE, Osong-eup (KR); Dae Young KIM, Sejong-si (KR); So Ra PARK, Sejong-si (KR)

(21) Appl. No.: 18/941,142

(22) Filed: Nov. 8, 2024

Related U.S. Application Data
(63) Continuation of application No. PCT/KR2023/006327, filed on May 10, 2023.

(30) Foreign Application Priority Data
May 10, 2022 (KR) 10-2022-0057475

(51) Int. Cl. C07K 16/40

(52) U.S. Cl. CPC C07K 16/40; C12Y 302/01014; C07K 2317/33; C07K 2317/92

(57) ABSTRACT

An anti-chitinase-3-like protein 1 antibody or antigen-binding fragment thereof of the present invention comprises a light chain variable region comprising CDRs of table 1 and a heavy chain variable region comprising CDRs of table 2. The antibody is capable of specifically binding to chitinase-3-like protein 1 (CHI3L1) and thus can be used for the prevention or treatment of a CHI3L1-associated disease, or for the diagnosis of the disease.

### CROSS-REFERENCE TO RELATED APPLICATIONS

This application is a continuation of International Application No. PCT/KR2023/006327 filed on May 10, 2023, which claims priority to Korean Patent Application No. 10-2022-0057475 filed on May 10, 2022, the entire contents of which are herein incorporated by reference.

### SEQUENCE LISTING

A computer-readable form (CRF) sequence listing having file name TWZ2010PC_seqlist.xml (208,552 bytes), created on Mar. 9, 2023, is incorporated herein by reference. The nucleic and amino acid sequences listed in the accompanying sequence listing are shown using standard abbreviations as defined in 37 C.F.R. § 1.822.

### TECHNICAL FIELD

The present invention relates to an anti-chitinase-3-like protein 1 antibody. More specifically, the present invention relates to an antibody capable of specifically binding to chitinase-3-like protein 1 (CHI3L1) and thus can be used for the prevention or treatment of a CHI3L1-associated disease, or for the diagnosis of the disease.

### BACKGROUND ART

Chitinase-3-like protein 1 (CHI3L1) is known to promote cancer metastasis, suppress immune function, and induce apoptosis inhibition, and is known to be significantly involved in the onset and progression of cancer diseases such as inflammatory responses and angiogenesis processes.

Although CHI3L1 has a low expression level under normal conditions, it has been reported that the expression level of CHI3L1 is significantly increased in the blood and tissues of patients with cancer or patients with inflammatory diseases, and thus its potential as a diagnostic marker for various types of diseases is emerging.

Accordingly, the present inventors sought to develop an antibody or antibody-related molecule that is capable of specifically binding to the above-described CHI3L1 and thus can be used for the prevention, treatment, or diagnosis of a CHI3L1-associated disease.

Related art documents include U.S. Pat. No. 7,670,599.

### SUMMARY

### Technical Problem

The main object of the present invention is to provide a novel antibody capable of specifically binding to chitinase-3-like protein 1 (CHI3L1).

Another object of the present invention is to provide a pharmaceutical composition for preventing or treating a CHI3L1-associated disease, a composition for diagnosing the disease, a kit for diagnosing the disease, and a method of providing information for diagnosing the disease by using the antibody.

Still another object of the present invention is to provide a polynucleotide, vector, and cell that can be used to produce the antibody.

### Technical Solution

According to one aspect of the present invention, the present invention provides an anti-chitinase-3-like protein 1 (CHI3L1) antibody or an antigen-binding fragment thereof, including a light chain variable region including CDR1 of SEQ ID NO: 1, CDR2 of SEQ ID NO: 2, and CDR3 of SEQ ID NO: 3, and a heavy chain variable region including CDR1 of SEQ ID NO: 31, CDR2 of SEQ ID NO: 32, and CDR3 of SEQ ID NO: 33; a light chain variable region including CDR1 of SEQ ID NO: 4, CDR2 of SEQ ID NO: 5, and CDR3 of SEQ ID NO: 6, and a heavy chain variable region including CDR1 of SEQ ID NO: 34, CDR2 of SEQ ID NO: 35, and CDR3 of SEQ ID NO: 36; a light chain variable region including CDR1 of SEQ ID NO: 7, CDR2 of SEQ ID NO: 8, and CDR3 of SEQ ID NO: 9, and a heavy chain variable region including CDR1 of SEQ ID NO: 37, CDR2 of SEQ ID NO: 38, and CDR3 of SEQ ID NO: 39; a light chain variable region including CDR1 of SEQ ID NO: 10, CDR2 of SEQ ID NO: 11, and CDR3 of SEQ ID NO: 12, and a heavy chain variable region including CDR1 of SEQ ID NO: 40, CDR2 of SEQ ID NO: 41, and CDR3 of SEQ ID NO: 42; a light chain variable region including CDR1 of SEQ ID NO: 13, CDR2 of SEQ ID NO: 14, and CDR3 of SEQ ID NO: 15, and a heavy chain variable region including CDR1 of SEQ ID NO: 43, CDR2 of SEQ ID NO: 44, and CDR3 of SEQ ID NO: 45; a light chain variable region including CDR1 of SEQ ID NO: 16, CDR2 of SEQ ID NO: 17, and CDR3 of SEQ ID NO: 18, and a heavy chain variable region including CDR1 of SEQ ID NO: 46, CDR2 of SEQ ID NO: 47, and CDR3 of SEQ ID NO: 48; a light chain variable region including CDR1 of SEQ ID NO: 19, CDR2 of SEQ ID NO: 20, and CDR3 of SEQ ID NO: 21, and a heavy chain variable region including CDR1 of SEQ ID NO: 49, CDR2 of SEQ ID NO: 50, and CDR3 of SEQ ID NO: 51; a light chain variable region including CDR1 of SEQ ID NO: 22, CDR2 of SEQ ID NO: 23, and CDR3 of SEQ ID NO: 24, and a heavy chain variable region including CDR1 of SEQ ID NO: 52, CDR2 of SEQ ID NO: 53, and CDR3 of SEQ ID NO: 54; a light chain variable region including CDR1 of SEQ ID NO: 25, CDR2 of SEQ ID NO: 26, and CDR3 of SEQ ID NO: 27, and a heavy chain variable region including CDR1 of SEQ ID NO: 55, CDR2 of SEQ ID NO: 56, and CDR3 of SEQ ID NO: 57; or a light chain variable region including CDR1 of SEQ ID NO: 28, CDR2 of SEQ ID NO: 29, and CDR3 of SEQ ID NO: 30, and a heavy chain variable region including CDR1 of SEQ ID NO: 58, CDR2 of SEQ ID NO: 59, and CDR3 of SEQ ID NO: 60.

According to another aspect of the present invention, the present invention provides a pharmaceutical composition for preventing or treating a CHI3L1-associated disease, including the antibody or antigen-binding fragment thereof of the present invention.

According to still another aspect of the present invention, the present invention provides a composition for diagnosing a CHI3L1-associated disease, including the antibody or antigen-binding fragment thereof of the present invention.

According to yet another aspect of the present invention, the present invention provides a kit for diagnosing a CHI3L1-associated disease, including the diagnostic composition of the present invention.

According to yet another aspect of the present invention, the present invention provides a method of providing information for diagnosing a CHI3L1-associated disease, including bringing the antibody or the antigen-binding fragment thereof into contact with a sample isolated from a test subject; and measuring a complex of the antibody or the antigen-binding fragment thereof and CHI3L1 formed by the contact.

According to yet another aspect of the present invention, the present invention provides a polynucleotide encoding the antibody or antigen-binding fragment thereof of the present invention.

According to yet another aspect of the present invention, the present invention provides a vector including the polynucleotide of the present invention.

According to yet another aspect of the present invention, the present invention provides a cell transformed with the vector of the present invention.

### Advantageous Effects

The antibody or antigen-binding fragment thereof of the present invention is capable of specifically binding to chitinase-3-like protein 1 (CHI3L1), and thus can be used for the prevention or treatment of a CHI3L1-associated disease, or for the diagnosis of the disease. Accordingly, by using the composition, kit, and information providing method of the present invention, a CHI3L1-associated disease can be effectively prevented, treated, or diagnosed. In addition, by using the polynucleotide, vector, and cell of the present invention, the antibody or antigen-binding fragment thereof of the present invention having the above-described excellent effects can be efficiently produced.

### DESCRIPTION OF DRAWINGS

FIG. 1A shows a schematic diagram illustrating a process of selecting antibodies exhibiting significant binding ability to human chitinase-3-like protein 1 (hCHI3L1) through phage display, and FIGS. 1B to 1D show the results of panning titration for Fab-I and Fab-II, respectively.

FIG. 2 shows the results of investigating the amino acid length distribution of the heavy chain CDR3 (HCDR3) of 10 antibodies against hCHI3L1 (A5, A7, D2, F2, E7, F4, H1, 1E12, C5, and H7) selected from phage display.

FIG. 3A shows the results of sodium dodecyl sulfate-polyacrylamide gel electrophoresis (SDS-PAGE) for confirming IgG production from 10 clones (A5, A7, D2, F2, E7, F4, H1, 1E12, C5, and H7) having antibodies against hCHI3L1, and FIG. 3B shows the results of measuring the amount of IgG production.

FIGS. 4A and 4B show the results of measuring EC50 values through enzyme-linked immunosorbent assay (ELISA) for 10 antibodies against hCHI3L1.

FIG. 5 shows the results of measuring the migrated cell number after treating A549 cell line with A5, A7, D2, F2, E7, F4, H1, 1E12, C5, and H7, which are antibodies against hCHI3L1, as candidate antibodies.

### DETAILED DESCRIPTION

The antibody or antigen-binding fragment thereof of the present invention, which was discovered through phage display panning performed by using chitinase-3-like protein 1 (CHI3L1) as an antigen, is capable of specifically binding to CHI3L1.

CHI3L1, also referred to as YKL-40, is known to be involved in various cancers, cancer metastasis, immune function, and apoptosis.

The present invention provides 10 types of antibodies or antigen-binding fragments thereof.

The 10 types are named ‘A5,’ ‘A7,’ ‘D2,’ ‘F2,’ ‘E7,’ ‘F4,’ ‘H1,’ ‘1E12,’ ‘C5,’ and ‘H7,’ as shown in Tables 1 to 4, and each type of antibody or antigen-binding fragment thereof has the amino acid sequences of CDR1, CDR2, and CDR3 of the light chain variable region shown in Table 1 (expressed in Table 1 as ‘CDRL1,’ ‘CDRL2,’ and ‘CDRL3,’ respectively, meaning complementarity-determining regions (CDRs) of the light chain variable region) and the amino acid sequences of CDR1, CDR2, and CDR3 of the heavy chain variable region shown in Table 2 (expressed in Table 2 as ‘CDRH1,’ ‘CDRH2,’ and ‘CDRH3,’ respectively, meaning CDRs of the heavy chain variable region).

In one embodiment, each type of antibody or antigen-binding fragment thereof has an amino acid sequence of the light chain variable region as shown in Table 3, and an amino acid sequence of the heavy chain variable region as shown in Table 4.

The antibody or antigen-binding fragment thereof of the present invention may be in various forms satisfying the conditions described above. For example, it may be an antibody selected from the group consisting of IgG, IgA, IgM, IgE, and IgD, and may be a fragment selected from the group consisting of fragment antigen binding (Fab), Fab′, F(ab′) 2, single-chain variable fragment (scFv), single-chain fragment variable-crystallizable fragment (scFv-Fc), a diabody, a minibody, and a triabody.

The antibody or antigen-binding fragment thereof of the present invention may be labeled for detection. At this time, labeling may be performed, for example, by direct or indirect binding of a detectable labeling substance.

It has been experimentally demonstrated that the antibody or antigen-binding fragment thereof of the present invention is capable of specifically binding to CHI3L1 with high binding affinity. Therefore, the antibody or antigen-binding fragment thereof of the present invention can be used to prevent or treat a CHI3L1-associated disease, and can also be used to diagnose a CHI3L1-associated disease.

Therefore, the present invention provides a pharmaceutical composition for preventing or treating a CHI3L1-associated disease, including the antibody or antigen-binding fragment thereof of the present invention.

In addition, the present invention provides a composition for diagnosing a CHI3L1-associated disease, including the antibody or antigen-binding fragment thereof of the present invention.

In addition, the present invention provides a kit for diagnosing a CHI3L1-associated disease, including the antibody or antigen-binding fragment thereof of the present invention or the diagnostic composition of the present invention.

In addition, the present invention provides a method of providing information for diagnosing a CHI3L1-associated disease, including: bringing the antibody or the antigen-binding fragment thereof into contact with a sample isolated from a test subject; and measuring a complex of the antibody or the antigen-binding fragment thereof and CHI3L1 formed by the contact.

In one embodiment of the present invention, a CHI3L1-associated disease is cancer, cancer metastasis, arthritis, or dementia, but is not limited thereto. In one embodiment, a CHI3L1-associated disease is lung cancer metastasis.

A pharmaceutical composition for preventing or treating a CHI3L1-associated disease of the present invention includes the above-described antibody or antigen-binding fragment thereof of the present invention. The pharmaceutical composition of the present invention may include one or more types of the antibody or antigen-binding fragment thereof of the present invention, and may include all ten types. The pharmaceutical composition of the present invention may be the antibody or antigen-binding fragment thereof of the present invention itself, or in a form in which the antibody or antigen-binding fragment thereof is mixed with a pharmaceutically acceptable carrier. The pharmaceutical composition of the present invention may be administered orally or parenterally during clinical administration, and may be used in the form of a general pharmaceutical preparation. The pharmaceutical composition of the present invention may be used alone or in combination with methods using surgery, radiotherapy, hormone therapy, chemotherapy, and biological response modifiers. The pharmaceutical composition of the present invention may be administered at the same dosage as a general antibody preparation based on the antibody or antigen-binding fragment thereof of the present invention contained therein. For example, the pharmaceutical composition may be administered in an amount of about 1 to 10 mg per 1 kg of body weight based on the antibody or antigen-binding fragment thereof of the present invention, but the range may vary depending on the weight, age, sex, health conditions, diet, administration time, administration method, excretion rate, and disease severity of the administration subject. When clinically administered, it may be formulated into various oral or parenteral dosage forms, and at this time, diluents or excipients such as fillers, extenders, binders, wetting agents, disintegrants, and surfactants may be used.

The composition for diagnosing a CHI3L1-associated disease of the present invention includes the above-described antibody or antigen-binding fragment thereof of the present invention. The diagnostic composition of the present invention may also, like the pharmaceutical composition, include one or more types of the antibody or antigen-binding fragment thereof of the present invention, and may include all ten types. The diagnostic composition of the present invention may include an antibody or an antigen-binding fragment thereof other than the antibody or antigen-binding fragment thereof of the present invention. In addition, a substance necessary for a binding reaction between the antibody or antigen-binding fragment thereof of the present invention and an antigen, such as a reagent, may be further included. In addition, a substance necessary for detecting the binding, a substance for stabilizing the antibody or antigen-binding fragment thereof of the present invention, and the like may be further included.

The kit for diagnosing a CHI3L1-associated disease of the present invention includes the diagnostic composition of the present invention. In addition to the diagnostic composition of the present invention, the diagnostic kit of the present invention may further include reagents, devices, and the like that are necessary for diagnosing a CHI3L1-associated disease using the diagnostic composition of the present invention, such as reagents and devices necessary for detecting binding of the antibody or antigen-binding fragment thereof of the present invention to an antigen.

The method for providing information diagnosing a CHI3L1-associated disease of the present invention includes bringing the antibody or antigen-binding fragment thereof of the present invention into contact with a sample isolated from a test subject; and measuring a complex of the antibody or the antigen-binding fragment thereof and CHI3L1 formed by the contact. In the information providing method of the present invention, the test subject may be a mammal, and is preferably a human. In the information providing method of the present invention, the sample may be a biological sample, for example, a sample of tissue, a cell, a body fluid, or the like isolated from the test subject, and may be blood or serum. In the information providing method of the present invention, the measurement of the complex may be achieved using a conventional method used to measure a binding complex between an antibody or antibody-related molecule and an antigen.

The present invention also provides a polynucleotide encoding the antibody or antigen-binding fragment thereof of the present invention.

In one embodiment, the polynucleotide of the present invention has the base sequences of the encoding regions of CDR1, CDR2, and CDR3 of the light chain variable region shown in Table 5 (expressed in Table 5 as ‘CDRL1,’ ‘CDRL2,’ and ‘CDRL3,’ meaning CDRs of the light chain variable region), and the base sequences of the encoding regions of CDR1, CDR2, and CDR3 of the heavy chain variable region shown in Table 6 (expressed in Table 6 as ‘CDRH1,’ ‘CDRH2,’ and ‘CDRH3,’ meaning CDRs of the heavy chain variable region).

In one embodiment, the polynucleotide encoding each type of the antibody or antigen-binding fragment thereof of the present invention has light chain variable region encoding base sequences shown in Table 7, and heavy chain variable region encoding base sequences shown in Table 8.

The present invention also provides a vector including the polynucleotide of the present invention. In one embodiment, the vector of the present invention is configured to express the antibody or antigen-binding fragment thereof encoded by the polynucleotide in a host cell. In one embodiment, the vector of the present invention includes an origin of replication, a promoter, a selectable marker, and the like suitable for replication and expression in a host cell.

The present invention also provides a cell transformed with the vector of the present invention. In one embodiment, the cell of the present invention is a cell capable of expressing the antibody or antigen-binding fragment thereof encoded by the polynucleotide included in the vector of the present invention, and a cell in which the vector of the present invention may be replicated.

The antibody or antigen-binding fragment thereof of the present invention may be produced using the amino acid sequence and base sequence information related to the antibody or the antigen-binding fragment thereof, the polynucleotide, the vector, or the cell of the present invention.

TABLE 1

| ANTIBODY | CDRL1 | CDRL2 | CDRL3 |
|---|---|---|---|
| A5 | RASQSISRWLN | ATSTLQS | QQSYSFPWT |
| | (SEQ ID NO: 1) | (SEQ ID NO: 2) | (SEQ ID NO: 3) |
| A7 | RASQDISNYLN | AASSLQS | QQSYSFPYT |
| | (SEQ ID NO: 4) | (SEQ ID NO: 5) | (SEQ ID NO: 6) |
| D2 | RASQSISRYLN | AASSLQS | QQSSSTPWT |
| | (SEQ ID NO: 7) | (SEQ ID NO: 8) | (SEQ ID NO: 9) |
| F2 | RASQDISSWLN | AASRLQS | QQAYSFPWT |
| | (SEQ ID NO: 10) | (SEQ ID NO: 11) | (SEQ ID NO: 12) |
| E7 | RASQSISNWLN | ATSRLQS | QQSYSFPYT |
| | (SEQ ID NO: 13) | (SEQ ID NO: 14) | (SEQ ID NO: 15) |
| F4 | RASQSISRWLN | AASRLQS | QQSYSTPWT |
| | (SEQ ID NO: 16) | (SEQ ID NO: 17) | (SEQ ID NO: 18) |
| H1 | RASQSISNWLN | ATSRLQS | QQSYSFPLT |
| | (SEQ ID NO: 19) | (SEQ ID NO: 20) | (SEQ ID NO: 21) |
| 1E12 | RASQSISNYLN | ATSTLHS | QQSYSFPWT |
| | (SBQ ID NO: 22) | (SEQ ID NO: 23) | (SEQ ID NO: 24) |
| C5 | RASQDISRWLN | AASTLQS | QQSYSFPLT |
| | (SEQ ID NO: 25) | (SEQ ID NO: 26) | (SEQ ID NO: 27) |
| H7 | RASQSISSWLN | AASRLQS | QQTYSFPWT |
| | (SEQ ID NO: 28) | (SEQ ID NO: 29) | (SEQ ID NO: 30) |

TABLE 2

| ANTIBODY | CDRH1 | CDRH2 | CDRH3 |
|---|---|---|---|
| A5 | SYGMS | AIKGSGGSTYYADSVKG | TYGGEIDY |
| | (SEQ ID NO: 31) | (SEQ ID NO: 32) | (SEQ ID NO: 33) |
| A7 | DYAMS | GISSSGGTIYYADSVKG | AIGSWADI |
| | (SEQ ID NO: 34) | (SEQ ID NO: 35) | (SEQ ID NO: 36) |
| D2 | SYAMH | AISSSSGTIYYADSVKG | DFGAVEDY |
| | (SEQ ID NO: 37) | (SEQ ID NO: 38) | (SEQ ID NO: 39) |
| F2 | SYAMH | SIKSSGGSKNYADSVKG | GYGASEDF |
| | (SEQ ID NO: 40) | (SEQ ID NO: 41) | (SEQ ID NO: 42) |
| E7 | SYAMH | AISSSGGSIYYADSVKG | QGLSFDI |
| | (SEQ ID NO: 43) | (SEQ ID NO: 44) | (SEQ ID NO: 45) |
| F4 | SYAMS | AISGSSGSTYYADSVKG | AQGTSADF |
| | (SEQ ID NO: 46) | (SEQ ID NO: 47) | (SEQ ID NO: 48) |
| H1 | SYAMS | AIRSSGGTTYYADSVKG | SQSTVADY |
| | (SEQ ID NO: 49) | (SEQ ID NO: 50) | (SEQ ID NO: 51) |
| 1E12 | SYAMH | AISSSGGTKYYADSVKG | GLQGGAGAFDI |
| | (SEQ ID NO: 52) | (SEQ ID NO: 53) | (SEQ ID NO: 54) |
| C5 | SYAMS | SISSSSGTTHYADSVKG | GGAFDY |
| | (SEQ ID NO: 55) | (SEQ ID NO: 56) | (SEQ ID NO: 57) |
| H7 | SYGMH | GISSSGGSKYYADSVKG | HGHQQMDF |
| | (SEQ ID NO: 58) | (SEQ ID NO: 59) | (SEQ ID NO: 60) |

TABLE 3

| | AMINO ACID SEQUENCE OF LIGHT CHAIN VARIABLE REGION OF ANTIBODY |
|---|---|
| ANTIBODY | (CDR: UNDERLINED) |
| A5 | DIQMTQSPSSLSASVGDRVTITCRASQSISRWLNWYQQKPGKAPKLLIY |
| | ATSTLQS GVPSRFSGSGSGTDFTLTISSLQPEDFATYYC QQSYSFPWT |
| | FGQGTKVEIK (SEQ ID NO: 61) |
| A7 | DIQMTQSPSSLSASVGDRVTITCRASQDISNYLNWYQQKPGKAPKLLIY |
| | AASSLQS GVPSRFSGSGSGTDFTLTISSLQPEDFATYYC QQSYSFPYT |
| | FGQGTKVEIK (SEQ ID NO: 62) |
| D2 | DIQMTQSPSSLSASVGDRVTITCRASQSISRYLNWYQQKPGKAPKLLIY |
| | AASSLQS GVPSRFSGSGSGTDFTLTISSLQPEDFATYYC QQSSSTPWT |
| | FGQGTKVEIK (SEQ ID NO: 63) |
| F2 | DIQMTQSPSSLSASVGDRVTITCRASQDISSWLNWYQQKPGKAPKLLIY |
| | AASRLQS GVPSRFSGSGSGTDFTLTISSLQPEDFATYYC QQAYSFPWT |
| | FGQGTKVEIK (SEQ ID NO: 64) |
| E7 | DIQMTQSPSSLSASVGDRVTITCRASQSISNWLNWYQQKPGKAPKLLIY |
| | ATSRLQS GVPSRFSGSGSGTDFTLTISSLQPEDFATYYC QQSYSFPYT |
| | FGQGTKVEIK (SEQ ID NO: 65) |
| F4 | DIQMTQSPSSLSASVGDRVTITCRASQSISRWLNWYQQKPGKAPKLLIY |
| | AASRLQS GVPSRFSGSGSGTDFTLTISSLQPEDFATYYC QQSYSTPWT |
| | FGQGTKVEIK (SEQ ID NO: 66) |
| H1 | DIQMTQSPSSLSASVGDRVTITCRASQSISNWLNWYQQKPGKAPKLLIY |
| | ATSRLQS GVPSRFSGSGSGTDFTLTISSLQPEDFATYYC QQSYSFPLT |
| | FGQGTKVEIK (SEQ ID NO: 67) |
| 1E12 | DIQMTQSPSSLSASVGDRVTITCRASQSISNYLNWYQQKPGKAPKLLIY |
| | ATSTLHS GVPSRFSGSGSGTDFTLTISSLQPEDFATYYC QQSYSFPWT |
| | FGQGTKVEIK (SEQ ID NO: 68) |
| C5 | DIQMTQSPSSLSASVGDRVTITCRASQDISRWLNWYQQKPGKAPKLLIY |
| | AASTLQS GVPSRESGSGSGTDFTLTISSLQPEDFATYYC QQSYSFPLT |
| | FGQGTKVEIK (SEQ ID NO: 69) |
| H7 | DIQMTQSPSSLSASVGDRVTITCRASQSISSWLNWYQQKPGKAPKLLIY |
| | AASRLQS GVPSRFSGSGSGTDFTLTISSLQPEDFATYYC QQTYSFPWT |
| | FGQGTKVEIK (SEQ ID NO: 70) |

TABLE 4

| | AMINO ACID SEQUENCE OF HEAVY CHAIN VARIABLE REGION OF ANTIBODY |
|---|---|
| ANTIBODY | (CDR: UNDERLINED) |
| A5 | EVQLVESGGGLVQPGGSLRLSCAASGFTFS SYGMS WVRQAPGKGLEWVS |
| | AIKGSGGSTYYADSVKG RFTISRDNSKNTLYLQMNSLRAEDTAVYYCAK |
| | TYGGFIDY WGQGTLVTVSS (SEQ ID NO: 71) |
| A7 | EVQLVESGGGLVQPGGSLRLSCAASGFTFS DYAMS WVRQAPGKGLEWVS |
| | GISSSGGTIYYADSVKG RFTISRDNSKNTLYLQMNSLRAEDTAVYYCAK |
| | AIGSWADI WGQGTLVTVSS (SEQ ID NO: 72) |
| D2 | EVQLVESGGGLVQPGGSLRLSCAASGFTFS SYAMHWVRQAPGKGLEWVS |
| | AISSSSGTIYYADSVKG RFTISRDNSKNTLYLQMNSLRAEDTAVYYCAK |
| | DFGAVFDY WGQGTLVTVSS (SEQ ID NO: 73) |
| F2 | EVQLVESGGGLAQPGGSLRLSCAASGFTFS SYAMHWVRQAPGKGLEWVS |
| | SIKSSGGSKNYADSVKG RFTISRDNSKNTLYLQMNSLRAEDTAVYYCAK |
| | GYGASFDF WGQGTLVTVSS (SEQ ID NO: 74) |
| E7 | EVQLVESGGGLVQPGGSLRLSCAASGFTFS SYAMHWVRQAPGKGLEWVS |
| | AISSSGGSIYYADSVKG RFTISRDNSKNTLYLQMNSLRAEDTAVYYCAK QGLSFDI |
| | WGQGTLVTVSS (SEQ ID NO: 75) |
| F4 | EVQLVESGGGLVQPGGSLRLSCAASGFTFS SYAMSWVRQAPGKGLEWVS |
| | AISGSSGSTYYADSVKG RFTISRDNSKNTLYLQMNSLRAEDTAVYYCAK |
| | AQGTSADF WGQGTLVTVSS (SEQ ID NO: 76) |
| H1 | EVQLVESGGGLVQPGGSLRLSCAASGFTFS SYAMSWVRQAPGKGLEWVS |
| | AIRSSGGITYYADSVKG RFTISRDNSKNTLYLQMNSLRAEDTAVYYCAK |
| | SQSTVADY WGQGTLVTVSS (SEQ ID NO: 77) |
| 1E12 | EVQLVESGGGLVQPGGSLRLSCAASGFTFS SYAMHWVRQAPGKGLEWVS |
| | AISSSGGTKYYADSVKG RFTISRDNSKNTLYLQMNSLRAEDTAVYYCAK |
| | GLQGGAGARDI WGQGTLVTVSS (SEQ ID NO: 78) |
| C5 | EVQLVESGGGLVQPGGSLRLSCAASGFTFS SYAMSWVRQAPGKGLEWVS |
| | SISSSSGTTHYADSVKGRFTISRDNSKNTLYLQMNSLRAEDTAVYYCAK GGAFDY |
| | WGQGTLVTVSS (SEQ ID NO: 79) |
| H7 | EVQLVESGGGLVQPGGSLRLSCAASGFTFS SYGMHWVRQAPGKGLEWVS |
| | GISSSGGSKYYADSVKGRFTISRDNSKNTLYLQMNSLRAEDTAVYYCAK |
| | RGHQQMDF WGQGTLVTVSS (SEQ ID) NO: 80) |

TABLE 5

| ANTIBODY | CDRL1 | CDRL2 | CDRL3 |
|---|---|---|---|
| A5 | CGCGCTAGCCAGTCTATCT | GCAACTTCCACTCTG | CAGCAATCTTACTCTTTTC |
| | CTCGTTGGCTGAAC | CAGTCT (SEQ ID NO: 82) | CGTGGACG (SEQ ID NO: 83) |
| | (SEQ ID NO: 81) | | |
| A7 | CGCGCTAGCCAGGATATCT | GCAGCATCCTCTCTG | CAGCAATCTTACTCTTTTC |
| | CTAATTACCTGAAC | CAGTCT (SEQ ID NO: 85) | CGTACACG (SEQ ID NO: 86) |
| | (SEQ ID NO: 84) | | |
| D2 | CGCGCTAGCCAGTCTATCT | GCAGCATCCTCTCTG | CAGCAATCTTCTTCTACTC |
| | CTCGTTACCTGAAC | CAGTCT (SEQ ID NO: 88) | CGTGGACG (SEQ ID NO: 89) |
| | (SEQ ID NO: 87) | | |
| F2 | CGCGCTAGCCAGGATATCT | GCAGCATCCCGTCTG | CAGCAAGCATACTCTTTTC |
| | CTTCTTGGCTGAAC | CAGTCT (SEQ ID NO: 91) | CGTGGACG (SEQ ID NO: 92) |
| | (SEQ ID NO: 90) | | |
| E7 | CGCGCTAGCCAGTCTATCT | GCAACTTCCCGTCTG | CAGCAATCTTACTCTTTTC |
| | CTAATTGGCTGAAC | CAGTCT (SEQ ID NO: 94) | CGTACACG (SEQ ID NO: 95) |
| | (SEQ ID NO: 93) | | |
| F4 | CGCGCTAGCCAGTCTATCT | GCAGCATCCCGTCTG | CAGCAATCTTACTCTACTC |
| | CTCGTTGGCTGAAC | CAGTCT (SEQ ID NO: 97) | CGTGGACG (SEQ ID NO: 98) |
| | (SEQ ID NO: 96) | | |
| H1 | CGCGCTAGCCAGTCTATCT | GCAACTTCCCGTCTG | CAGCAATCTTACTCTTTTC |
| | CTAATTGGCTGAAC | CAGTCT (SEQ ID NO: 100) | CGCTGACG (SEQ ID NO: 101) |
| | (SEQ ID NO: 99) | | |
| 1E12 | CGCGCTAGCCAGTCTATCT | GCAACTTCCACTCTG | CAGCAATCTTACTCTTTTC |
| | CTAATTACCTGAAC | CACTCT (SEQ ID NO: 103) | CGTGGACG (SEQ ID NO: 104) |
| | (SEQ ID NO: 102) | | |
| C5 | CGCGCTAGCCAGGATATCT | GCAGCATCCACTCTG | CAGCAATCTTACTCTTTTC |
| | CTCGTTGGCTGAAC | CAGTCT (SEQ ID NO: 106) | CGCTGACG (SEQ ID NO: 107) |
| | (SEQ ID NO: 105) | | |
| H7 | CGCGCTAGCCAGTCTATCT | GCAGCATCCCGTCTG | CAGCAAACTTACTCTTTTC |
| | CTTCTTGGCTGAAC | CAGTCT (SEQ ID NO: 109) | CGTGGACG (SEQ ID NO: 110) |
| | (SEQ ID NO: 108) | | |

TABLE 6

| ANTIBODY | CDRH1 | CDRH2 | CDRH3 |
|---|---|---|---|
| A5 | TCTTATGGTATG | GCAATCAAAGGTTCTGGTGGTTC | ACTTACGGTGGTGAAAT |
| | TCT (SEQ ID NO: 111) | TACTTACTATGCCGATTCAGTGA | CGATTAC (SEQ ID NO: 113) |
| | | AGGGT (SEQ ID NO: 112) | |
| A7 | GATTATGCAATG | GGTATCTCTTCTTCTGGTGGTACT | GCAATCGGTTCTTGGGC |
| | TCT | ATCTACTATGCCGATTCAGTGAA | AGATATC (SEQ ID NO: 116) |
| | (SEQ ID NO: 114) | GGGT (SEQ ID NO: 115) | |
| D2 | TCTTATGCAATG | GCAATCTCTTCTTCTTCTGGTACT | GATTTCGGTGCAGTTTT |
| | CAC (SEQ ID NO: 117) | ATCTACTATGCCGATTCAGTGAA | CGATTAC (SEQ ID NO: 119) |
| | | GGGT (SEQ ID NO: 118) | |
| F2 | TCTTATGCAATG | TCTATCAAATCTTCTGGTGGTTC | GGTTACGGTGCATCTTT |
| | CAC (SEQ ID NO: 120) | TAAAAATTATGCCGATTCAGTGA | CGATTTC (SEQ ID NO: 122) |
| | | AGGGT (SEQ ID NO: 121) | |
| E7 | TCTTATGCAATG | GCAATCTCTTCTTCTGGTGGTTCT | CAGGGTCTGTCTTTCGA |
| | CAC (SEQ ID NO: 123) | ATCTACTATGCCGATTCAGTGAA | TATC (SEQ ID NO: 125) |
| | | GGGT (SEQ ID NO: 124) | |
| F4 | TCTTATGGAATG | GCAATCTCTGGTTCTTCTGGTTCT | GCACAGGGTACTTCTGC |
| | TCT (SEQ ID NO: 126) | ACTTACTATGCCGATTCAGTGAA | AGATTTC (SEQ ID NO: 128) |
| | | GGGT (SEQ ID NO: 127) | |
| H1 | TCTTATGCAATG | GCAATCCGTTCTTCTGGTGGTACT | TCTCAGTCTACTGTTGC |
| | TCT (SEQ ID NO: 129) | ACTTACTATGCCGATTCAGTGAA | AGATTAC (SEQ ID NO: 131) |
| | | GGGT (SEQ ID NO: 130) | |
| 1E12 | TCTTATGCAATG | GCAATCTCTTCTTCTGGTGGTACT | GGTCTGCAGGGTGGTGC |
| | CAC (SEQ ID NO: 132) | AAATACTATGCCGATTCAGTGAA | AGGTGCATTTGATATC |
| | | GGGT (SEQ ID NO: 133) | (SEQ ID NO: 134) |
| C5 | TCTTATGCAATG | TCTATCTCTTCTTCTTCTGGTACT | GGTGGTGCATTCGATTA |
| | TCT (SEQ ID NO: 135) | ACTCACTATGCCGATTCAGTGAA | C (SEQ ID NO: 137) |
| | | GGGT (SEQ ID NO: 136) | |
| H7 | TCTTATGGTATG | GGTATCTCTTCTTCTGGTGGTTCT | CATGGTCATCAGCAGAT |
| | CAC (SEQ ID NO: 138) | AAATACTATGCCGATTCAGTGAA | GGATTTC (SEQ ID NO: 140) |
| | | GGGT (SEQ ID NO: 139) | |

TABLE 7

| ANTIBODY | BASE SEQUENCE OF LIGHT CHAIN VARIABLE REGION OF ANTIBODY (CDR: UNDERLINED) |
|---|---|
| A5 | GACATTCAAATGACGCAGAGTCCCTCCTCACTGAGTGCTAGCGTGGGCGATCGTGTGACAATTACTT |
| | GTCGCGCTAGCCAGTCTATCTCTCGTTGGCTGAACTGGTATCAGCAGAAACCGGGCAAGGCGCCAAA |
| | ATTGCTGATTTACGCAACTTCCACTCTGCAGTCTGGTGTACCGTCCCGTTTCTCTGGCAGGGGTTCTG |
| | GTACGGATTTTACCCTGACCATAAGCCTCCAGCCTGAAGATTTTGCCACCTATTATTGTCAGCA |
| | ATCTTACTCCGTGGACGTTCGGGCAGGGAACTAAAGTGGAAATTAAA (SEQ ID NO: 141) |
| A7 | GACATTCAAATGACGCAGAGTCCCTCCTCACTGAGTGCTAGCGTGGGCGATCGTGTGACAATTACTT |
| | GTCGCGCTAGCCAGGATATCTCTAATTACCTGAACTGGTATCAGCAGAAACCGGGCAAGGCGCCAAA |
| | ATTGCTGATTTACGCAGCATCCTCTCTGCAGTCTGGTGTACCGTCCCGTTTCTCTGGCAGCGGTTCTG |
| | GTACGGATTTTACCCTGACCATCTCAAGCCTCCAGCCTGAAGATTTTGCCACCTATTATTGTCAGCA |
| | ATCTTACTCTTTTCCGTACACGTTCGGGCAGGGAACTAAAGTGGAAATTAAA (SEQ ID NO: 142) |
| D2 | GACATTCAAATGACGCAGAGTCCCTCCTCACTGAGTGCTAGCGTGGGCGATCGTGTGACAATTACTT |
| | GTCGCGCTAGCCAGTCTATCTCTCGTTACCTGAACTGGTATCAGCAGAAACCGGGCAAGGCGCCAAA |
| | ATTGCTGATTTACGCAGCATCCTCTCTGCAGTCTGGTGTACCGTCCCGTTTCTCTGGCAGCGGTTCTG |
| | GTACGGATTTTACCCTGACCATCTCAAGCCTCCAGCCTGAAGATTTTGCCACCTATTATTGTCAGCA |
| | ATCTTCTTCTACTCCGTGGAACGTTCGGGCAGGGAACTAAAGTGGAAATTAAA (SEQ ID NO: 143) |
| F2 | GACATTCAAATGACGCAGAGTCCCTCCTCACTGAGTGCTAGCGTGGGCGATCGTGTGACAATTACTT |
| | GTCGCGCTAGCCAGGATATCTCTTCTTGGCTGAACTGGTATCAGCAGAAACCGGGCAAGGCGCCAAA |
| | ATTGCTGATTTACGCAGCATCCCGTCTGCAGTCTGGTGTACCGTCCCGTTTCTCGGCAGCGGTTCTG |
| | GTACGATTTTACCTGACCATCTCAAGCCTCCAGCCTGAAGATTTTGCCACCTATTATTGTCAGCA |
| | AGCATACTCTTTTCCGTGGACGTTCGGGCAGGGAACTAAAGTGGAAATTAAA (SEQ ID NO: 144) |
| E7 | GACATTCAAATGACGCAGAGTCCCTCCTCACTGAGTGCTAGCGTGGGGGATCGTGTGACAATTACTT |
| | GTCGCGCTAGCCAGTCTATCTCTAATTGGCTGAACTGGTATCAGCAGAAACCGGGCAAGGCGCCAAA |
| | ATTGCTGATTTACGCAACTTCCCGTCTGCAGTCTGGTGTACCGTCCCGTTTCTCTGGCAGCGGTTCTG |
| | GTACGGATTTTACCCTGACCATCTCAAGCCTCCAGCCTGAAGATTTTGCCACCTATTATTGTCAGCA |
| | ATCTTACTCTTTTCCGTACACGTTCGGGCAGGGAACTAAAGTGGAAATTAAA (SEQ ID NO: 145) |
| F4 | GACATTCAAATGACGCAGAGTCCCTCCTCACTGAGTGCTAGCGTGGGCGATCGTGTGACAATTACTT |
| | GTCGCGCTAGCCAGTCTATCTCTCGTTGGCTGAACTGGTATCAGCAGAAACCGGGCAAGGCGCCAAA |
| | ATTGCTGATTTACGCAGCATCCCGTCTGCAGTCTGGTGTACCGTCCCGTTTCTCTGGCAGCGGTTCTG |
| | GTACGGATTTTACCCTGACCATCTCAAGCCTCCAGCCTGAAGATTTTGCCACCTATTATTGTCAGCA |
| | ATCTTACTTCTACTCCGTGGACGTTCGGGCAGGGAACTAAAGTGGAAATTAAA (SEQ ID NO: 146) |
| H1 | GACATTCAAATGACGCAGAGTCCCTCCTCACTGAGTGCTAGCGTGGGCGATCGTGTGACAATTACTT |
| | GTCGCGCTAGCCAGTCTATCTCTAATTGGCTGAACTGGTATCAGCAGAAACCGGGCAAGGCGCCAAA |
| | ATTGCTGATTTACGCAACTTCCCGTCTGCAGTCTGGTGTACCGTCCCGTTTCTCTGGCAGCGGTTCTG |
| | GTACGGATTTTACCCTGACCATCTCAAGCCTCGAGCCTGAAGATTTTGCCACCTATTATTGTCAGCA |
| | ATCTTACTCTTTTCCGCTGACGTTCGGGCAGGGAACTAAAGTGGAAATTAAA (SEQ ID NO: 147) |
| 1E12 | GACATTCAAATGACCCAGAGTCCCTCCTCACTGAGTGCTAGCGTGGGGGATCGTGTGACAATTACTT |
| | GTCGCGCTAGCCAGTCTATCTCTAATTACCTGAACTGGTATCAGCAGAAACCGGGCAAGGCGCGAAÅ |
| | ATTGCTGATTTACGCAACTTCCACTCTGCACTCTGGTGTACCGTCCCGTTTCTCTGGCAGCGGTTCTG |
| | GTACGGATTTTACCCTGACCATCTCAAGCCTCCAGCCTGAAGATTTTGCCACCTATTATTGTCAGCA |
| | ATCTTACTCTTTTCCGTGGACGTTCGGGCAGGGAACTAAAGTGGAAATTAAA (SEQ ID NO: 148) |
| C5 | GACATTCAAATGACGCAGAGTCCCTCCTCACTGAGTGCTAGCGTGGGCGATCGTGTGACAATTACTT |
| | GTCGCGCTAGCCAGGATATCTCTCGTTGGCTGAACTGGTATCAGCAGAAACCGGGCAAGGCGCCAAA |
| | ATTGCTGATTTACGCAGCATCCACTCTGCAGTCTGGTGTACCGTCCCGTTTCTCTGGCAGCGGTTCTG |
| | GTACGGATTTTACCCTGACCATCTGAAGCCTCCAGCCTGAAGATTTTGCCACCTATTATTGTCAGCA |
| | ATCTTACTCTTTTCCGCTGACGTTCGGGCAGGGAACTAAAGTGGAAATTAAA (SEQ ID NO: 149) |
| H7 | GACATTCAAATGACGCAGAGTCCCTCCTCACTGAGTGCTAGCGTGGGCGATCGTGTGACAATTACTT |
| | GTCGCGCTAGCCAGTCTATCTCTTCTTGGCTGAACTGGTATCAGCAGAAACCGGGCAAGGCGCCAAA |
| | ATTGCTGATTTACGCAGCATCCCGTCTGCAGTCTGGTGTACCGTCCCGTTTCTCTGGCAGCGGTTCTG |
| | GTACGGATTTTACCCTGACCATCTCAAGCCTCCAGCCTGAAGATTTTGCCACCTATTATTGTCAGCA |
| | AACTTACTCTTTTCCGTGGACGTTCGGGCAGGGAACTAAAGTGGAAATTAAA (SEQ ID NO: 150) |

TABLE 8

| ANTIBODY | BASE SEQUENCE OF HEAVY CHAIN VARIABLE REGION OF ANTIBODY (CDR: UNDERLINED) |
|---|---|
| A5 | GAAGTACAGTTGGTCGAAAGTGGCGGTGGCCTCGTGCAACCGGGTGGTTCACTGCGTCTGAGCTGCGC |
| | CGCCTCGGGTTTTACTTTCTCTTCTTATGGTATGTCTTGGGTTCGTCAGGGGCCGGGCAAGGGTCTCG |
| | AATGGGTTTCAGCAATCAAAGGTTCTGGTGGTTCTACTTACTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTCTATTATTGCGCCAAAACTTACGGTGGTGAAATCGATTACTGGGGTCAGGGCACTTTAG |
| | TGACCGTCTCATCG (SEQ ID NO: 151) |
| A7 | GAAGTACAGTTGGTCGAAAGTGGCGGTGGCCTCGTGCAACCGGGTGGTTCACTGCGTCTGAGCTGCGC |
| | CGCCTCGGGTTTTACTTTCTCTGATTATGCAATGTCTTGGGTTCGTCAGGCGCCGGGCAAGGGTCTCG |
| | AATGGGTTTCAGGTATCTCTTCTTCTGGTGGTACTATCTACTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTCTATTATTGCGCCAAAGCAATCGGTTCTTGGGCAGATATCTGGGGTCAGGGCACTTTAG |
| | TGACCGTCTCATCG (SEQ ID NO: 152) |
| D2 | GAAGTACAGTTGGTCGAAAGTGGCGGTGGCCTCGTGCAACCGGGTGGTTCACTGCGTCTGAGCTGCGC |
| | CGCCTCGGGTTTTACTTTCTCTTCTTATGCAATGCACTGGGTTCGTCAGGGGCCGGGCAAGGGTCTCG |
| | AATGGGTTTCAGCAATCTCTTCTTCTTCTGGTACTATCTACTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTGTATTATTGCGCCAAAGATTTCGGTGGAGTTTTCGATTACTGGGGTCAGGGCACTTTAG |
| | TGACCGTCTCATCG (SEQ ID NO: 153) |
| F2 | GAAGTACAGTTGGTCGAAAGTGGCGGTGGCCTCGCGCAACCGGGTGGTTCACTGCGTGTGAGCTGCGC |
| | CGCCTCGGGTTTTACTTTCTCTTCTTATGCAATGCACTGGGTTCGTCAGGCGCCGGGCAAGGGTCTCG |
| | AATGGGTTTCATCTATCAAATCTTCTGGTGGTTCTAAAAATTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTCTATTATTGCGCCAAAGGTTACGGTGCATCTTTCGATTTCTGGGGTCAGGGCACTTTAG |
| | TGACCGTCTCATCG (SEQ ID NO: 154) |
| E7 | GAAGTACAGTTGGTCGAAAGTGGCGGTGGCCTCGTGCAACCGGGTGGTTCACTGCGTCTGAGGTGCGC |
| | CGCCTCGGGTTTTACTTTCTCTTCTTATGCAATGCACTGGGTTCGTCAGGCGCGGGGCAAGGGTCTCG |
| | AATGGGTTTCAGCAATCTCTTCTTCTGGTGGTTCTATCTACTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTCTATTATTGCGCCAAACAGGGTCTGTCTTTCGATATCTGGGGTCAGGGCACTTTAGTGAC |
| | CGTCTCATCG (SEQ ID NO: 155) |
| F4 | GAAGTACAGTTGGTCGAAAGTGGCGGTGGCCTCGTGCAACCGGGTGGTTCACTGCGTCTGAGCTGCGC |
| | CGCCTCGGGTTTTACTTTCTCTTCTTATGCAATGTCTTGGGTTCGTCAGGCGCCGGGCAAGGGTCTCG |
| | AATGGGTTTCAGCAATCTCTGGTTCTTCTGGTTCTACTTACTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTCTATTATTGCGCCAAAGCACAGGGTACTTCTGCAGATTTCTGGGGTCAGGGCACTTTAGT |
| | GACCGTCTCATCG (SEQ ID NO: 156) |
| H1 | GAAGTACAGTTGGTCGAAAGTGGCGGTGGCCTCGTGCAACCGGGTGGTTCACTGCGTCTGAGCTGCGC |
| | CGCCTCGGGTTTTACTTTCTCTTCTTATGCAATGTCTTGGGTTCGTCAGGCGCCGGGCAAGGGTCTCG |
| | AATGGGTTTCAGCAATCCGTTCTTCTGGTGGTACTACTTACTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTCTATTATTGCGCCAAATCTCAGTCTACTGTTGCAGATTACTGGGGTCAGGGCACTTTAG |
| | TGACCGTCTCATCG (SEQ ID NO: 157) |
| 1E12 | GAAGTACAGTTGGTCGAAAGTGGCGGTGGCCTCGTGCAACCGGGTGGTTCACTGCGTCTGAGCTGCGC |
| | CGCCTCGGGTTTTACTTTCTCTTCTTATGCAATGCACTGGGTTCGTCAGGCGCCGGGCAAGGGTCTCG |
| | AATGGGTTTCAGCAATCTCTTCTTCTGGTGGTACTAAATACTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTCTATTATTGCGCCAAAGGTCTGCAGGGTGGTGCAGGTGCATTTGATATCTGGGGTCAGGG |
| | TACTCTGGTTACTGTATCATCG (SEQ ID NO: 158) |
| C5 | GAAGTACAGTTGGTCGAAAGTGGCGGTGGCCTCGTGCAACCGGGTGGTTCACTGCGTCTGAGCTGCGC |
| | CGCCTCGGGTTTTACTTTCTCTTCTTATGCAATGTCTTGGGTTCGTCAGGCGCCGGGCAAGGGTCTCG |
| | AATGGGTTTCATCTATCTCTTCTTCTTCTGGTACTACTCACTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTCTATTATTGCGCCAAAGGTGGTGGATTCGATTACTGGGGTCAGGGCACTTTAGTGACCGT |
| | CTCATCG (SEQ ID NO: 159) |
| H7 | GAAGTACAGTTGGTCGAAAGTGGCGGTGGCCTCGTGCAACCGGGTGGTTCACTGCGTCTGAGCTGCGC |
| | CGCCTCGGGTTTTACTTTCTCTTCTTATGGTATGCACTGGGTTCGTCAGGCGCCGGGCAAGGGTCTCG |
| | AATGGGTTTCAGGTATCTCTTCTTCTGGTGGTTCTAAATACTATGCCGATTCAGTGAAGGGTCGCTT |
| | TACCATTTCCCGTGACAACTCTAAGAATACTCTGTATCTGCAGATGAACTCGCTGCGTGCCGAAGAC |
| | ACGGCCGTCTATTATTGCGCCAAACATGGTCATCAGCAGATGGATTTCTGGGGTCAGGGCACTTTAG |
| | TGACCGTCTCATCG (SEQ ID NO: 160) |

Hereinafter, the present invention will be described in more detail through examples. Since these examples are only intended to illustrate the present invention, the scope of the present invention is not to be construed as being limited by these examples.

### EXAMPLES

### Example 1. Screening of Fab Antibody Clones Against CHI3L1

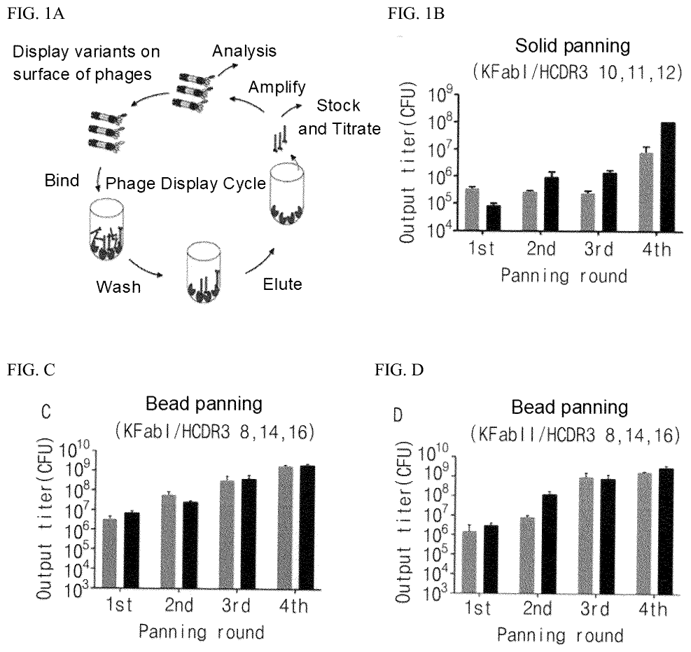

To discover antibodies that specifically bind to CHI3L1, immunotube and magnetic bead-based phage display panning was performed using CHI3L1 as an antigen, and monoclonal phage ELISA was performed to select clones that specifically bind to CHI3L1 (FIG. 1).

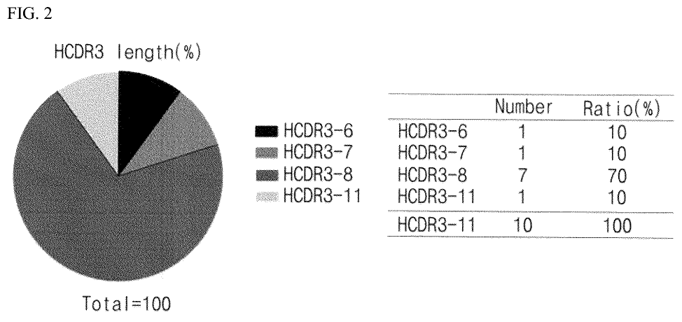

A total of 10 Fab clones were selected through base sequence analysis and amino acid sequence analysis of the clones that were found to be positive in the monoclonal phage ELISA. As a result of investigating the length distribution of the heavy chain CDR3 (HCDR3) of the selected Fab clones, it was confirmed that 70% of the clones had eight residues, and 10% of the clones respectively had 6, 7, and 11 residues (FIG. 2).

### Example 2. Expression and Purification of Antibodies in the Form of IgG

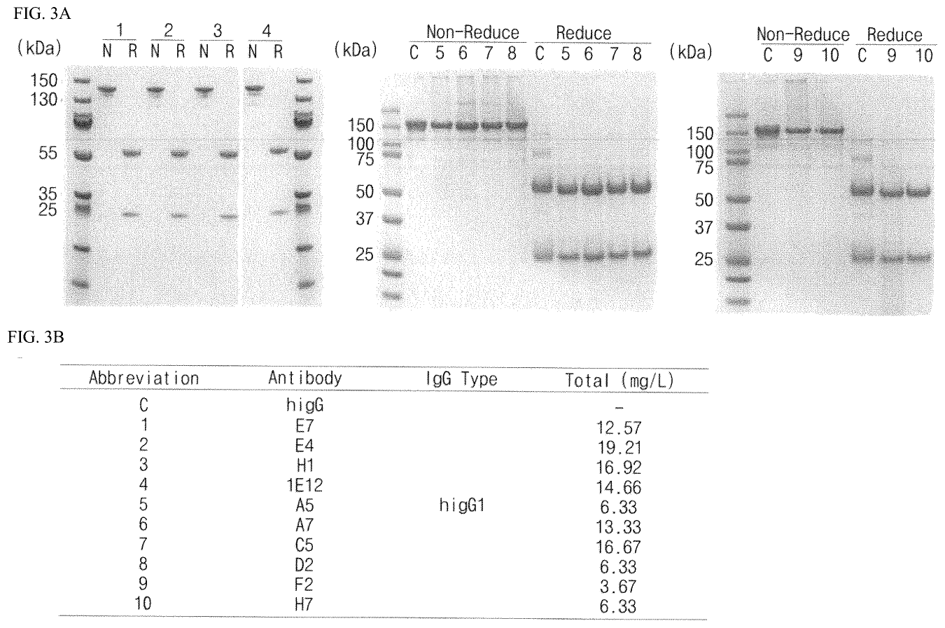

The clones selected in Example 1 were converted into the IgG form of human antibodies, and an experiment was performed to verify the results. Ten types of anti-CHI3L1 antibodies were co-transfected into ExpiCHO-S cells and cultured, and IgG was purified using a Protein A resin column. The molecular weight and purity of the purified IgG antibodies were confirmed through SDS-PAGE (FIG. 3).

### Example 3. ELISA Analysis of Antibodies

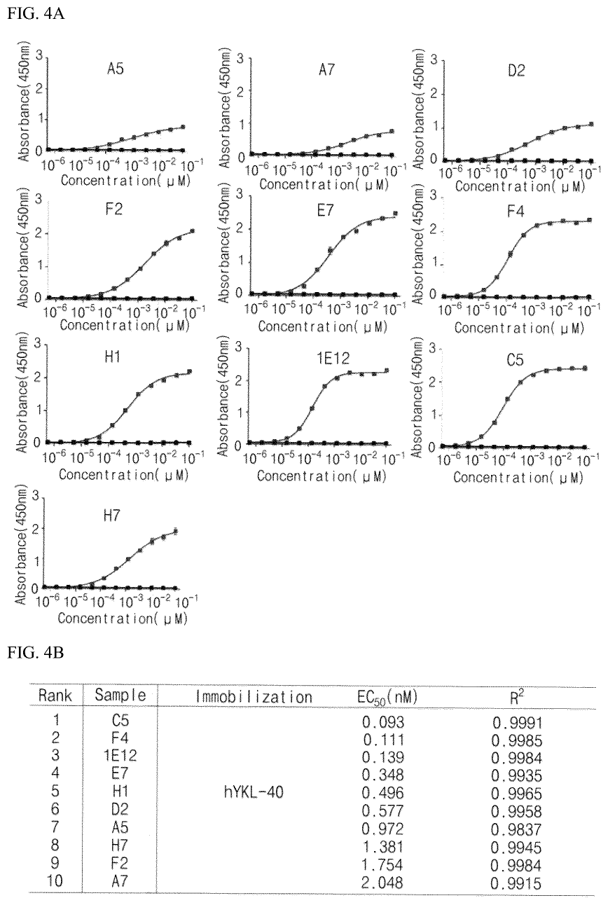

An experiment was performed to measure antigen affinity by investigating the EC50 value for each antibody through ELISA. As a result, it was confirmed that all 10 antibodies bound to the CHI3L1 antigen protein. In terms of the EC50 value, C5, F4, and 1E12 antibodies exhibited particularly excellent binding ability (FIG. 4).

### Example 4. Analysis of Anticancer Efficacy of Antibodies

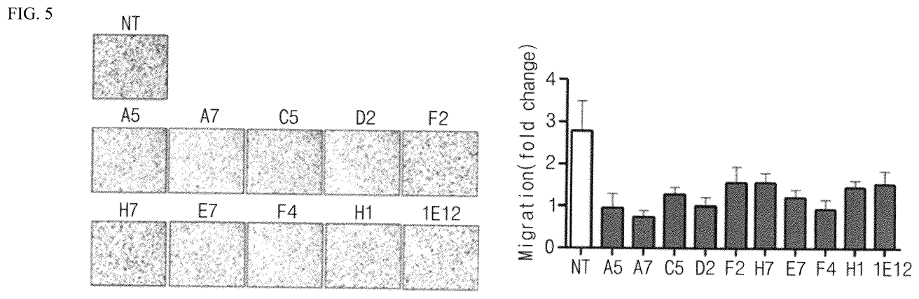

An experiment was performed to determine whether antibodies against CHI3L1 had an effect of inhibiting cancer metastasis when lung cancer cell lines were treated. The migration of the human lung cancer cell line, A549 cell line, was quantitatively investigated in a permeable insert. As a result, it was confirmed that when the 10 antibodies against human CHI3L1 (hCHI3L1) were treated, the number of migrated cells in the A549 cell line decreased compared to the control group (FIG. 5).

What is claimed is:

1. An anti-chitinase-3-like protein 1 antibody or an antigen-binding fragment thereof, comprising:
a light chain variable region including CDR1 of SEQ ID NO: 1, CDR2 of SEQ ID NO: 2, and CDR3 of SEQ ID NO: 3, and a heavy chain variable region including CDR1 of SEQ ID NO: 31, CDR2 of SEQ ID NO: 32, and CDR3 of SEQ ID NO: 33;
a light chain variable region including CDR1 of SEQ ID NO: 4, CDR2 of SEQ ID NO: 5, and CDR3 of SEQ ID NO: 6, and a heavy chain variable region including CDR1 of SEQ ID NO: 34, CDR2 of SEQ ID NO: 35, and CDR3 of SEQ ID NO: 36;
a light chain variable region including CDR1 of SEQ ID NO: 7, CDR2 of SEQ ID NO: 8, and CDR3 of SEQ ID NO: 9, and a heavy chain variable region including CDR1 of SEQ ID NO: 37, CDR2 of SEQ ID NO: 38, and CDR3 of SEQ ID NO: 39;
a light chain variable region including CDR1 of SEQ ID NO: 10, CDR2 of SEQ ID NO: 11, and CDR3 of SEQ ID NO: 12, and a heavy chain variable region including CDR1 of SEQ ID NO: 40, CDR2 of SEQ ID NO: 41, and CDR3 of SEQ ID NO: 42;
a light chain variable region including CDR1 of SEQ ID NO: 13, CDR2 of SEQ ID NO: 14, and CDR3 of SEQ ID NO: 15, and a heavy chain variable region including CDR1 of SEQ ID NO: 43, CDR2 of SEQ ID NO: 44, and CDR3 of SEQ ID NO: 45;
a light chain variable region including CDR1 of SEQ ID NO: 16, CDR2 of SEQ ID NO: 17, and CDR3 of SEQ ID NO: 18, and a heavy chain variable region including CDR1 of SEQ ID NO: 46, CDR2 of SEQ ID NO: 47, and CDR3 of SEQ ID NO: 48;
a light chain variable region including CDR1 of SEQ ID NO: 19, CDR2 of SEQ ID NO: 20, and CDR3 of SEQ ID NO: 21, and a heavy chain variable region including CDR1 of SEQ ID NO: 49, CDR2 of SEQ ID NO: 50, and CDR3 of SEQ ID NO: 51;
a light chain variable region including CDR1 of SEQ ID NO: 22, CDR2 of SEQ ID NO: 23, and CDR3 of SEQ ID NO: 24, and a heavy chain variable region including CDR1 of SEQ ID NO: 52, CDR2 of SEQ ID NO: 53, and CDR3 of SEQ ID NO: 54;
a light chain variable region including CDR1 of SEQ ID NO: 25, CDR2 of SEQ ID NO: 26, and CDR3 of SEQ ID NO: 27, and a heavy chain variable region including CDR1 of SEQ ID NO: 55, CDR2 of SEQ ID NO: 56, and CDR3 of SEQ ID NO: 57; or
a light chain variable region including CDR1 of SEQ ID NO: 28, CDR2 of SEQ ID NO: 29, and CDR3 of SEQ ID NO: 30, and a heavy chain variable region including CDR1 of SEQ ID NO: 58, CDR2 of SEQ ID NO: 59, and CDR3 of SEQ ID NO: 60.

2. The antibody or the antigen-binding fragment thereof of claim 1, comprising:
a light chain variable region of SEQ ID NO: 61 and a heavy chain variable region of SEQ ID NO: 71;
a light chain variable region of SEQ ID NO: 62 and a heavy chain variable region of SEQ ID NO: 72;
a light chain variable region of SEQ ID NO: 63 and a heavy chain variable region of SEQ ID NO: 73;
a light chain variable region of SEQ ID NO: 64 and a heavy chain variable region of SEQ ID NO: 74;
a light chain variable region of SEQ ID NO: 65 and a heavy chain variable region of SEQ ID NO: 75;
a light chain variable region of SEQ ID NO: 66 and a heavy chain variable region of SEQ ID NO: 76;
a light chain variable region of SEQ ID NO: 67 and a heavy chain variable region of SEQ ID NO: 77;
a light chain variable region of SEQ ID NO: 68 and a heavy chain variable region of SEQ ID NO: 78;
a light chain variable region of SEQ ID NO: 69 and a heavy chain variable region of SEQ ID NO: 79; or
a light chain variable region of SEQ ID NO: 70 and a heavy chain variable region of SEQ ID NO: 80.

3. A pharmaceutical composition for preventing or treating a chitinase-3-like protein 1-associated disease, comprising the antibody or the antigen-binding fragment thereof of claim 1.

4. The pharmaceutical composition of claim 3, wherein the chitinase-3-like protein 1-associated disease is cancer, cancer metastasis, arthritis, or dementia.

5. The pharmaceutical composition of claim 4, wherein the chitinase-3-like protein 1-associated disease is lung cancer metastasis.

6. A composition for diagnosing a chitinase-3-like protein 1-associated disease, comprising the antibody or the antigen-binding fragment thereof of claim 1.

7. The composition of claim 6, wherein the chitinase-3-like protein 1-associated disease is cancer, cancer metastasis, arthritis, or dementia.

8. A kit for diagnosing a chitinase-3-like protein 1-associated disease, comprising the composition of claim 6.

9. The kit of claim 8, wherein the chitinase-3-like protein 1-associated disease is cancer, cancer metastasis, arthritis, or dementia.

10. A method of providing information for diagnosing a chitinase-3-like protein 1-associated disease, comprising:
bringing the antibody or the antigen-binding fragment thereof of claim 1 into contact with a sample isolated from a test subject; and
measuring a complex of the antibody or the antigen-binding fragment thereof and chitinase-3-like protein 1 formed by the contact.

11. The method of claim 10, wherein the chitinase-3-like protein 1-associated disease is cancer, cancer metastasis, arthritis, or dementia.

12. A polynucleotide encoding the antibody or the antigen-binding fragment thereof of claim 1.

13. The polynucleotide of claim 12, comprising:
a light chain variable region encoding sequence including a CDR1 encoding sequence of SEQ ID NO: 81, a CDR2 encoding sequence of SEQ ID NO: 82, and a CDR3 encoding sequence of SEQ ID NO: 83, and a heavy chain variable region encoding sequence including a CDR1 encoding sequence of SEQ ID NO: 111, a CDR2 encoding sequence of SEQ ID NO: 112, and a CDR3 encoding sequence of SEQ ID NO: 113;
a light chain variable region encoding sequence including a CDR1 encoding sequence of SEQ ID NO: 84, a CDR2 encoding sequence of SEQ ID NO: 85, and a CDR3 encoding sequence of SEQ ID NO: 86, and a heavy chain variable region encoding sequence including a CDR1 encoding sequence of SEQ ID NO: 114, a CDR2 encoding sequence of SEQ ID NO: 115, and a CDR3 encoding sequence of SEQ ID NO: 116;
a light chain variable region encoding sequence including a CDR1 encoding sequence of SEQ ID NO: 87, a CDR2 encoding sequence of SEQ ID NO: 88, and a CDR3 encoding sequence of SEQ ID NO: 89, and a heavy chain variable region encoding sequence including a CDR1 encoding sequence of SEQ ID NO: 117, a CDR2 encoding sequence of SEQ ID NO: 118, and a CDR3 encoding sequence of SEQ ID NO: 119;
a light chain variable region encoding sequence including a CDR1 encoding sequence of SEQ ID NO: 90, a CDR2 encoding sequence of SEQ ID NO: 91, and a CDR3 encoding sequence of SEQ ID NO: 92, and a heavy chain variable region encoding sequence including a CDR1 encoding sequence of SEQ ID NO: 120, a CDR2 encoding sequence of SEQ ID NO: 121, and a CDR3 encoding sequence of SEQ ID NO: 122;
a light chain variable region encoding sequence including a CDR1 encoding sequence of SEQ ID NO: 93, a CDR2 encoding sequence of SEQ ID NO: 94, and a CDR3 encoding sequence of SEQ ID NO: 95, and a heavy chain variable region encoding sequence including a CDR1 encoding sequence of SEQ ID NO: 123, a CDR2 encoding sequence of SEQ ID NO: 124, and a CDR3 encoding sequence of SEQ ID NO: 125;
a light chain variable region encoding sequence including a CDR1 encoding sequence of SEQ ID NO: 96, a CDR2 encoding sequence of SEQ ID NO: 97, and a CDR3 encoding sequence of SEQ ID NO: 98, and a heavy chain variable region encoding sequence including a CDR1 encoding sequence of SEQ ID NO: 126, a CDR2 encoding sequence of SEQ ID NO: 127, and a CDR3 encoding sequence of SEQ ID NO: 128;
a light chain variable region encoding sequence including a CDR1 encoding sequence of SEQ ID NO: 99, a CDR2 encoding sequence of SEQ ID NO: 100, and a CDR3 encoding sequence of SEQ ID NO: 101, and a heavy chain variable region encoding sequence including a CDR1 encoding sequence of SEQ ID NO: 129, a CDR2 encoding sequence of SEQ ID NO: 130, and a CDR3 encoding sequence of SEQ ID NO: 131;
a light chain variable region encoding sequence including a CDR1 encoding sequence of SEQ ID NO: 102, a CDR2 encoding sequence of SEQ ID NO: 103, and a CDR3 encoding sequence of SEQ ID NO: 104, and a heavy chain variable region encoding sequence including a CDR1 encoding sequence of SEQ ID NO: 132, a CDR2 encoding sequence of SEQ ID NO: 133, and a CDR3 encoding sequence of SEQ ID NO: 134;
a light chain variable region encoding sequence including a CDR1 encoding sequence of SEQ ID NO: 105, a CDR2 encoding sequence of SEQ ID NO: 106, and a CDR3 encoding sequence of SEQ ID NO: 107, and a heavy chain variable region encoding sequence including a CDR1 encoding sequence of SEQ ID NO: 135, a CDR2 encoding sequence of SEQ ID NO: 136, and a CDR3 encoding sequence of SEQ ID NO: 137; or
a light chain variable region encoding sequence including a CDR1 encoding sequence of SEQ ID NO: 108, a CDR2 encoding sequence of SEQ ID NO: 109, and a CDR3 encoding sequence of SEQ ID NO: 110, and a heavy chain variable region encoding sequence including a CDR1 encoding sequence of SEQ ID NO: 138, a CDR2 encoding sequence of SEQ ID NO: 139, and a CDR3 encoding sequence of SEQ ID NO: 140.

14. The polynucleotide of claim 13, comprising:
a light chain variable region encoding sequence of SEQ ID NO: 141 and a heavy chain variable region encoding sequence of SEQ ID NO: 151;
a light chain variable region encoding sequence of SEQ ID NO: 142 and a heavy chain variable region encoding sequence of SEQ ID NO: 152;
a light chain variable region encoding sequence of SEQ ID NO: 143 and a heavy chain variable region encoding sequence of SEQ ID NO: 153;
a light chain variable region encoding sequence of SEQ ID NO: 144 and a heavy chain variable region encoding sequence of SEQ ID NO: 154;
a light chain variable region encoding sequence of SEQ ID NO: 145 and a heavy chain variable region encoding sequence of SEQ ID NO: 155;
a light chain variable region encoding sequence of SEQ ID NO: 146 and a heavy chain variable region encoding sequence of SEQ ID NO: 156;
a light chain variable region encoding sequence of SEQ ID NO: 147 and a heavy chain variable region encoding sequence of SEQ ID NO: 157;
a light chain variable region encoding sequence of SEQ ID NO: 148 and a heavy chain variable region encoding sequence of SEQ ID NO: 158;
a light chain variable region encoding sequence of SEQ ID NO: 149 and a heavy chain variable region encoding sequence of SEQ ID NO: 159; or
a light chain variable region encoding sequence of SEQ ID NO: 150 and a heavy chain variable region encoding sequence of SEQ ID NO: 160.

15. A vector comprising the polynucleotide of claim 12.

16. A cell transformed with the vector of claim 15.


SEQUENCE LISTING (160 sequences)

SEQ ID NO: 1 - protein, 11 aa, synthetic construct
  REGION 1..11: note = CDRL1
RASQSISRWLN

SEQ ID NO: 2 - protein, 7 aa, synthetic construct
  REGION 1..7: note = CDRL2
ATSTLQS

SEQ ID NO: 3 - protein, 9 aa, synthetic construct
  REGION 1..9: note = CDRL3
QQSYSFPWT

SEQ ID NO: 4 - protein, 11 aa, synthetic construct
  REGION 1..11: note = CDRL1
RASQDISNYLN

SEQ ID NO: 5 - protein, 7 aa, synthetic construct
  REGION 1..7: note = CDRL2
AASSLQS

SEQ ID NO: 6 - protein, 9 aa, synthetic construct
  REGION 1..9: note = CDRL3
QQSYSFPYT

SEQ ID NO: 7 - protein, 11 aa, synthetic construct
  REGION 1..11: note = CDRL1
RASQSISRYLN

SEQ ID NO: 8 - protein, 7 aa, synthetic construct
  REGION 1..7: note = CDRL2
AASSLQS

SEQ ID NO: 9 - protein, 9 aa, synthetic construct
  REGION 1..9: note = CDRL3
QQSSSTPWT

SEQ ID NO: 10 - protein, 11 aa, synthetic construct
  REGION 1..11: note = CDRL1
RASQDISSWLN

SEQ ID NO: 11 - protein, 7 aa, synthetic construct
  REGION 1..7: note = CDRL2
AASRLQS

SEQ ID NO: 12 - protein, 9 aa, synthetic construct
  REGION 1..9: note = CDRL3
QQAYSFPWT

SEQ ID NO: 13 - protein, 11 aa, synthetic construct
  REGION 1..11: note = CDRL1
RASQSISNWLN

SEQ ID NO: 14 - protein, 7 aa, synthetic construct
  REGION 1..7: note = CDRL2
ATSRLQS

SEQ ID NO: 15 - protein, 9 aa, synthetic construct
  REGION 1..9: note = CDRL3
QQSYSFPYT

SEQ ID NO: 16 - protein, 11 aa, synthetic construct
  REGION 1..11: note = CDRL1
RASQSISRWLN

SEQ ID NO: 17 - protein, 7 aa, synthetic construct
  REGION 1..7: note = CDRL2
AASRLQS

SEQ ID NO: 18 - protein, 9 aa, synthetic construct
  REGION 1..9: note = CDRL3
QQSYSTPWT

SEQ ID NO: 19 - protein, 11 aa, synthetic construct
  REGION 1..11: note = CDRL1
RASQSISNWLN

SEQ ID NO: 20 - protein, 7 aa, synthetic construct
  REGION 1..7: note = CDRL2
ATSRLQS

SEQ ID NO: 21 - protein, 9 aa, synthetic construct
  REGION 1..9: note = CDRL3
QQSYSFPLT

SEQ ID NO: 22 - protein, 11 aa, synthetic construct
  REGION 1..11: note = CDRL1
RASQSISNYLN

SEQ ID NO: 23 - protein, 7 aa, synthetic construct
  REGION 1..7: note = CDRL2
ATSTLHS

SEQ ID NO: 24 - protein, 9 aa, synthetic construct
  REGION 1..9: note = CDRL3
QQSYSFPWT

SEQ ID NO: 25 - protein, 11 aa, synthetic construct
  REGION 1..11: note = CDRL1
RASQDISRWLN

SEQ ID NO: 26 - protein, 7 aa, synthetic construct
  REGION 1..7: note = CDRL2
AASTLQS

SEQ ID NO: 27 - protein, 9 aa, synthetic construct
  REGION 1..9: note = CDRL3
QQSYSFPLT

SEQ ID NO: 28 - protein, 11 aa, synthetic construct
  REGION 1..11: note = CDRL1
RASQSISSWLN

SEQ ID NO: 29 - protein, 7 aa, synthetic construct
  REGION 1..7: note = CDRL2
AASRLQS

SEQ ID NO: 30 - protein, 9 aa, synthetic construct
  REGION 1..9: note = CDRL3
QQTYSFPWT

SEQ ID NO: 31 - protein, 5 aa, synthetic construct
  REGION 1..5: note = CDRH1
SYGMS

SEQ ID NO: 32 - protein, 17 aa, synthetic construct
  REGION 1..17: note = CDRH2
AIKGSGGSTYYADSVKG

SEQ ID NO: 33 - protein, 8 aa, synthetic construct
  REGION 1..8: note = CDRH3
TYGGEIDY

SEQ ID NO: 34 - protein, 5 aa, synthetic construct
  REGION 1..5: note = CDRH1
DYAMS

SEQ ID NO: 35 - protein, 17 aa, synthetic construct
  REGION 1..17: note = CDRH2
GISSSGGTIYYADSVKG

SEQ ID NO: 36 - protein, 8 aa, synthetic construct
  REGION 1..8: note = CDRH3
AIGSWADI

SEQ ID NO: 37 - protein, 5 aa, synthetic construct
  REGION 1..5: note = CDRH1
SYAMH

SEQ ID NO: 38 - protein, 17 aa, synthetic construct
  REGION 1..17: note = CDRH2
AISSSSGTIYYADSVKG

SEQ ID NO: 39 - protein, 8 aa, synthetic construct
  REGION 1..8: note = CDRH3
DFGAVFDY

SEQ ID NO: 40 - protein, 5 aa, synthetic construct
  REGION 1..5: note = CDRH1
SYAMH

SEQ ID NO: 41 - protein, 17 aa, synthetic construct
  REGION 1..17: note = CDRH2
SIKSSGGSKNYADSVKG

SEQ ID NO: 42 - protein, 8 aa, synthetic construct
  REGION 1..8: note = CDRH3
GYGASFDF

SEQ ID NO: 43 - protein, 5 aa, synthetic construct
  REGION 1..5: note = CDRH1
SYAMH

SEQ ID NO: 44 - protein, 17 aa, synthetic construct
  REGION 1..17: note = CDRH2
AISSSGGSIYYADSVKG

SEQ ID NO: 45 - protein, 7 aa, synthetic construct
  REGION 1..7: note = CDRH3
QGLSFDI

SEQ ID NO: 46 - protein, 5 aa, synthetic construct
  REGION 1..5: note = CDRH1
SYAMS

SEQ ID NO: 47 - protein, 17 aa, synthetic construct
  REGION 1..17: note = CDRH2
AISGSSGSTYYADSVKG

SEQ ID NO: 48 - protein, 8 aa, synthetic construct
  REGION 1..8: note = CDRH3
AQGTSADF

SEQ ID NO: 49 - protein, 5 aa, synthetic construct
  REGION 1..5: note = CDRH1
SYAMS

SEQ ID NO: 50 - protein, 17 aa, synthetic construct
  REGION 1..17: note = CDRH2
AIRSSGGTTYYADSVKG

SEQ ID NO: 51 - protein, 8 aa, synthetic construct
  REGION 1..8: note = CDRH3
SQSTVADY

SEQ ID NO: 52 - protein, 5 aa, synthetic construct
  REGION 1..5: note = CDRH1
SYAMH

SEQ ID NO: 53 - protein, 17 aa, synthetic construct
  REGION 1..17: note = CDRH2
AISSSGGTKYYADSVKG

SEQ ID NO: 54 - protein, 11 aa, synthetic construct
  REGION 1..11: note = CDRH3
GLQGGAGAFDI

SEQ ID NO: 55 - protein, 5 aa, synthetic construct
  REGION 1..5: note = CDRH1
SYAMS

SEQ ID NO: 56 - protein, 17 aa, synthetic construct
  REGION 1..17: note = CDRH2
SISSSSGTTHYADSVKG

SEQ ID NO: 57 - protein, 6 aa, synthetic construct
  REGION 1..6: note = CDRH3
GGAFDY

SEQ ID NO: 58 - protein, 5 aa, synthetic construct
  REGION 1..5: note = CDRH1
SYGMH

SEQ ID NO: 59 - protein, 17 aa, synthetic construct
  REGION 1..17: note = CDRH2
GISSSGGSKYYADSVKG

SEQ ID NO: 60 - protein, 8 aa, synthetic construct
  REGION 1..8: note = CDRH3
HGHQQMDF

SEQ ID NO: 61 - protein, 107 aa, synthetic construct
  REGION 1..107: note = VL
DIQMTQSPSSLSASVGDRVTITCRASQSISRWLNWYQQKPGKAPKLLIYATSTLQSGVPSRFSGSGSGTDFTLTISSLQPEDFATYYCQQSYSFPWTFGQGTKVEIK

SEQ ID NO: 62 - protein, 107 aa, synthetic construct
  REGION 1..107: note = VL
DIQMTQSPSSLSASVGDRVTITCRASQDISNYLNWYQQKPGKAPKLLIYAASSLQSGVPSRFSGSGSGTDFTLTISSLQPEDFATYYCQQSYSFPYTFGQGTKVEIK

SEQ ID NO: 63 - protein, 107 aa, synthetic construct
  REGION 1..107: note = VL
DIQMTQSPSSLSASVGDRVTITCRASQSISRYLNWYQQKPGKAPKLLIYAASSLQSGVPSRFSGSGSGTDFTLTISSLQPEDFATYYCQQSSSTPWTFGQGTKVEIK

SEQ ID NO: 64 - protein, 107 aa, synthetic construct
  REGION 1..107: note = VL
DIQMTQSPSSLSASVGDRVTITCRASQDISSWLNWYQQKPGKAPKLLIYAASRLQSGVPSRFSGSGSGTDFTLTISSLQPEDFATYYCQQAYSFPWTFGQGTKVEIK

SEQ ID NO: 65 - protein, 107 aa, synthetic construct
  REGION 1..107: note = VL
DIQMTQSPSSLSASVGDRVTITCRASQSISNWLNWYQQKPGKAPKLLIYATSRLQSGVPSRFSGSGSGTDFTLTISSLQPEDFATYYCQQSYSFPYTFGQGTKVEIK

SEQ ID NO: 66 - protein, 107 aa, synthetic construct
  REGION 1..107: note = VL
DIQMTQSPSSLSASVGDRVTITCRASQSISRWLNWYQQKPGKAPKLLIYAASRLQSGVPSRFSGSGSGTDFTLTISSLQPEDFATYYCQQSYSTPWTFGQGTKVEIK

SEQ ID NO: 67 - protein, 107 aa, synthetic construct
  REGION 1..107: note = VL
DIQMTQSPSSLSASVGDRVTITCRASQSISNWLNWYQQKPGKAPKLLIYATSRLQSGVPSRFSGSGSGTDFTLTISSLQPEDFATYYCQQSYSFPLTFGQGTKVEIK

SEQ ID NO: 68 - protein, 107 aa, synthetic construct
  REGION 1..107: note = VL
DIQMTQSPSSLSASVGDRVTITCRASQSISNYLNWYQQKPGKAPKLLIYATSTLHSGVPSRFSGSGSGTDFTLTISSLQPEDFATYYCQQSYSFPWTFGQGTKVEIK

SEQ ID NO: 69 - protein, 107 aa, synthetic construct
  REGION 1..107: note = VL
DIQMTQSPSSLSASVGDRVTITCRASQDISRWLNWYQQKPGKAPKLLIYAASTLQSGVPSRFSGSGSGTDFTLTISSLQPEDFATYYCQQSYSFPLTFGQGTKVEIK

SEQ ID NO: 70 - protein, 107 aa, synthetic construct
  REGION 1..107: note = VL
DIQMTQSPSSLSASVGDRVTITCRASQSISSWLNWYQQKPGKAPKLLIYAASRLQSGVPSRFSGSGSGTDFTLTISSLQPEDFATYYCQQTYSFPWTFGQGTKVEIK

SEQ ID NO: 71 - protein, 117 aa, synthetic construct
  REGION 1..117: note = VH
EVQLVESGGGLVQPGGSLRLSCAASGFTFSSYGMSWVRQAPGKGLEWVSAIKGSGGSTYYADSVKGRFTISRDNSKNTLYLQMNSLRAEDTAVYYCAKTYGGEIDYWGQGTLVTVSS

SEQ ID NO: 72 - protein, 117 aa, synthetic construct
  REGION 1..117: note = VH
EVQLVESGGGLVQPGGSLRLSCAASGFTFSDYAMSWVRQAPGKGLEWVSGISSSGGTIYYADSVKGRFTISRDNSKNTLYLQMNSLRAEDTAVYYCAKAIGSWADIWGQGTLVTVSS

SEQ ID NO: 73 - protein, 117 aa, synthetic construct
  REGION 1..117: note = VH
EVQLVESGGGLVQPGGSLRLSCAASGFTFSSYAMHWVRQAPGKGLEWVSAISSSSGTIYYADSVKGRFTISRDNSKNTLYLQMNSLRAEDTAVYYCAKDFGAVFDYWGQGTLVTVSS

SEQ ID NO: 74 - protein, 117 aa, synthetic construct
  REGION 1..117: note = VH
EVQLVESGGGLAQPGGSLRLSCAASGFTFSSYAMHWVRQAPGKGLEWVSSIKSSGGSKNYADSVKGRFTISRDNSKNTLYLQMNSLRAEDTAVYYCAKGYGASFDFWGQGTLVTVSS

SEQ ID NO: 75 - protein, 116 aa, synthetic construct
  REGION 1..116: note = VH
EVQLVESGGGLVQPGGSLRLSCAASGFTFSSYAMHWVRQAPGKGLEWVSAISSSGGSIYYADSVKGRFTISRDNSKNTLYLQMNSLRAEDTAVYYCAKQGLSFDIWGQGTLVTVSS

SEQ ID NO: 76 - protein, 117 aa, synthetic construct
  REGION 1..117: note = VH
EVQLVESGGGLVQPGGSLRLSCAASGFTFSSYAMSWVRQAPGKGLEWVSAISGSSGSTYYADSVKGRFTISRDNSKNTLYLQMNSLRAEDTAVYYCAKAQGTSADFWGQGTLVTVSS

SEQ ID NO: 77 - protein, 117 aa, synthetic construct
  REGION 1..117: note = VH
EVQLVESGGGLVQPGGSLRLSCAASGFTFSSYAMSWVRQAPGKGLEWVSAIRSSGGTTYYADSVKGRFTISRDNSKNTLYLQMNSLRAEDTAVYYCAKSQSTVADYWGQGTLVTVSS

SEQ ID NO: 78 - protein, 120 aa, synthetic construct
  REGION 1..120: note = VH
EVQLVESGGGLVQPGGSLRLSCAASGFTFSSYAMHWVRQAPGKGLEWVSAISSSGGTKYYADSVKGRFTISRDNSKNTLYLQMNSLRAEDTAVYYCAKGLQGGAGAFDIWGQGTLVTVSS

SEQ ID NO: 79 - protein, 115 aa, synthetic construct
  REGION 1..115: note = VH
EVQLVESGGGLVQPGGSLRLSCAASGFTFSSYAMSWVRQAPGKGLEWVSSISSSSGTTHYADSVKGRFTISRDNSKNTLYLQMNSLRAEDTAVYYCAKGGAFDYWGQGTLVTVSS

SEQ ID NO: 80 - protein, 117 aa, synthetic construct
  REGION 1..117: note = VH
EVQLVESGGGLVQPGGSLRLSCAASGFTFSSYGMHWVRQAPGKGLEWVSGISSSGGSKYYADSVKGRFTISRDNSKNTLYLQMNSLRAEDTAVYYCAKHGHQQMDFWGQGTLVTVSS

SEQ ID NO: 81 - DNA, 33 bp, synthetic construct
  misc_feature 1..33: note = CDRL1
  source 1..33: mol_type = other DNA
cgcgctagccagtctatctctcgttggctgaac

SEQ ID NO: 82 - DNA, 21 bp, synthetic construct
  misc_feature 1..21: note = CDRL2
  source 1..21: mol_type = other DNA
gcaacttccactctgcagtct

SEQ ID NO: 83 - DNA, 27 bp, synthetic construct
  misc_feature 1..27: note = CDRL3
  source 1..27: mol_type = other DNA
cagcaatcttactcttttccgtggacg

SEQ ID NO: 84 - DNA, 33 bp, synthetic construct
  misc_feature 1..33: note = CDRL1
  source 1..33: mol_type = other DNA
cgcgctagccaggatatctctaattacctgaac

SEQ ID NO: 85 - DNA, 21 bp, synthetic construct
  misc_feature 1..21: note = CDRL2
  source 1..21: mol_type = other DNA
gcagcatcctctctgcagtct

SEQ ID NO: 86 - DNA, 27 bp, synthetic construct
  misc_feature 1..27: note = CDRL3
  source 1..27: mol_type = other DNA
cagcaatcttactcttttccgtacacg

SEQ ID NO: 87 - DNA, 33 bp, synthetic construct
  misc_feature 1..33: note = CDRL1
  source 1..33: mol_type = other DNA
cgcgctagccagtctatctctcgttacctgaac

SEQ ID NO: 88 - DNA, 21 bp, synthetic construct
  misc_feature 1..21: note = CDRL2
  source 1..21: mol_type = other DNA
gcagcatcctctctgcagtct

SEQ ID NO: 89 - DNA, 27 bp, synthetic construct
  misc_feature 1..27: note = CDRL3
  source 1..27: mol_type = other DNA
cagcaatcttcttctactccgtggacg

SEQ ID NO: 90 - DNA, 33 bp, synthetic construct
  misc_feature 1..33: note = CDRL1
  source 1..33: mol_type = other DNA
cgcgctagccaggatatctcttcttggctgaac

SEQ ID NO: 91 - DNA, 21 bp, synthetic construct
  misc_feature 1..21: note = CDRL2
  source 1..21: mol_type = other DNA
gcagcatcccgtctgcagtct

SEQ ID NO: 92 - DNA, 27 bp, synthetic construct
  misc_feature 1..27: note = CDRL3
  source 1..27: mol_type = other DNA
cagcaagcatactcttttccgtggacg

SEQ ID NO: 93 - DNA, 33 bp, synthetic construct
  misc_feature 1..33: note = CDRL1
  source 1..33: mol_type = other DNA
cgcgctagccagtctatctctaattggctgaac

SEQ ID NO: 94 - DNA, 21 bp, synthetic construct
  misc_feature 1..21: note = CDRL2
  source 1..21: mol_type = other DNA
gcaacttcccgtctgcagtct

SEQ ID NO: 95 - DNA, 27 bp, synthetic construct
  misc_feature 1..27: note = CDRL3
  source 1..27: mol_type = other DNA
cagcaatcttactcttttccgtacacg

SEQ ID NO: 96 - DNA, 33 bp, synthetic construct; identical to SEQ ID NO: 81
  misc_feature 1..33: note = CDRL1
  source 1..33: mol_type = other DNA

SEQ ID NO: 97 - DNA, 21 bp, synthetic construct
  misc_feature 1..21: note = CDRL2
  source 1..21: mol_type = other DNA
gcagcatcccgtctgcagtct

SEQ ID NO: 98 - DNA, 27 bp, synthetic construct
  misc_feature 1..27: note = CDRL3
  source 1..27: mol_type = other DNA
cagcaatcttactctactccgtggacg

SEQ ID NO: 99 - DNA, 33 bp, synthetic construct; identical to SEQ ID NO: 93
  misc_feature 1..33: note = CDRL1
  source 1..33: mol_type = other DNA

SEQ ID NO: 100 - DNA, 21 bp, synthetic construct
  misc_feature 1..21: note = CDRL2
  source 1..21: mol_type = other DNA
gcaacttcccgtctgcagtct

SEQ ID NO: 101 - DNA, 27 bp, synthetic construct
  misc_feature 1..27: note = CDRL3
  source 1..27: mol_type = other DNA
cagcaatcttactcttttccgctgacg

SEQ ID NO: 102 - DNA, 33 bp, synthetic construct
  misc_feature 1..33: note = CDRL1
  source 1..33: mol_type = other DNA
cgcgctagccagtctatctctaattacctgaac

SEQ ID NO: 103 - DNA, 21 bp, synthetic construct
  misc_feature 1..21: note = CDRL2
  source 1..21: mol_type = other DNA
gcaacttccactctgcactct

SEQ ID NO: 104 - DNA, 27 bp, synthetic construct
  misc_feature 1..27: note = CDRL3
  source 1..27: mol_type = other DNA
cagcaatcttactcttttccgtggacg

SEQ ID NO: 105 - DNA, 33 bp, synthetic construct
  misc_feature 1..33: note = CDRL1
  source 1..33: mol_type = other DNA
cgcgctagccaggatatctctcgttggctgaac

SEQ ID NO: 106 - DNA, 21 bp, synthetic construct
  misc_feature 1..21: note = CDRL2
  source 1..21: mol_type = other DNA
gcagcatccactctgcagtct

SEQ ID NO: 107 - DNA, 27 bp, synthetic construct
  misc_feature 1..27: note = CDRL3
  source 1..27: mol_type = other DNA
cagcaatcttactcttttccgctgacg

SEQ ID NO: 108 - DNA, 33 bp, synthetic construct
  misc_feature 1..33: note = CDRL1
  source 1..33: mol_type = other DNA
cgcgctagccagtctatctcttcttggctgaac

SEQ ID NO: 109 - DNA, 21 bp, synthetic construct
  misc_feature 1..21: note = CDRL2
  source 1..21: mol_type = other DNA
gcagcatcccgtctgcagtct

SEQ ID NO: 110 - DNA, 27 bp, synthetic construct
  misc_feature 1..27: note = CDRL3
  source 1..27: mol_type = other DNA
cagcaaacttactcttttccgtggacg

SEQ ID NO: 111 - DNA, 15 bp, synthetic construct
  misc_feature 1..15: note = CDRH1
  source 1..15: mol_type = other DNA
tcttatggtatgtct

SEQ ID NO: 112 - DNA, 51 bp, synthetic construct
  misc_feature 1..51: note = CDRH2
  source 1..51: mol_type = other DNA
gcaatcaaaggttctggtggttctacttactatgccgattcagtgaagggt

SEQ ID NO: 113 - DNA, 24 bp, synthetic construct
  misc_feature 1..24: note = CDRH3
  source 1..24: mol_type = other DNA
acttacggtggtgaaatcgattac

SEQ ID NO: 114 - DNA, 15 bp, synthetic construct
  misc_feature 1..15: note = CDRH1
  source 1..15: mol_type = other DNA
gattatgcaatgtct

SEQ ID NO: 115 - DNA, 51 bp, synthetic construct
  misc_feature 1..51: note = CDRH2
  source 1..51: mol_type = other DNA
ggtatctcttcttctggtggtactatctactatgccgattcagtgaagggt

SEQ ID NO: 116 - DNA, 24 bp, synthetic construct
  misc_feature 1..24: note = CDRH3
  source 1..24: mol_type = other DNA
gcaatcggttcttgggcagatatc

SEQ ID NO: 117 - DNA, 15 bp, synthetic construct
  misc_feature 1..15: note = CDRH1
  source 1..15: mol_type = other DNA
tcttatgcaatgcac

SEQ ID NO: 118 - DNA, 51 bp, synthetic construct
  misc_feature 1..51: note = CDRH2
  source 1..51: mol_type = other DNA
gcaatctcttcttcttctggtactatctactatgccgattcagtgaagggt

SEQ ID NO: 119 - DNA, 24 bp, synthetic construct
  misc_feature 1..24: note = CDRH3
  source 1..24: mol_type = other DNA
gatttcggtgcagttttcgattac

SEQ ID NO: 120 - DNA, 15 bp, synthetic construct
  misc_feature 1..15: note = CDRH1
  source 1..15: mol_type = other DNA
tcttatgcaatgcac

SEQ ID NO: 121 - DNA, 51 bp, synthetic construct
  misc_feature 1..51: note = CDRH2
  source 1..51: mol_type = other DNA
tctatcaaatcttctggtggttctaaaaattatgccgattcagtgaagggt

SEQ ID NO: 122 - DNA, 24 bp, synthetic construct
  misc_feature 1..24: note = CDRH3
  source 1..24: mol_type = other DNA
ggttacggtgcatctttcgatttc

SEQ ID NO: 123 - DNA, 15 bp, synthetic construct
  misc_feature 1..15: note = CDRH1
  source 1..15: mol_type = other DNA
tcttatgcaatgcac

SEQ ID NO: 124 - DNA, 51 bp, synthetic construct
  misc_feature 1..51: note = CDRH2
  source 1..51: mol_type = other DNA
gcaatctcttcttctggtggttctatctactatgccgattcagtgaagggt

SEQ ID NO: 125 - DNA, 21 bp, synthetic construct
  misc_feature 1..21: note = CDRH3
  source 1..21: mol_type = other DNA
cagggtctgtctttcgatatc

SEQ ID NO: 126 - DNA, 15 bp, synthetic construct
  misc_feature 1..15: note = CDRH1
  source 1..15: mol_type = other DNA
tcttatgcaatgtct

SEQ ID NO: 127 - DNA, 51 bp, synthetic construct
  misc_feature 1..51: note = CDRH2
  source 1..51: mol_type = other DNA
gcaatctctggttcttctggttctacttactatgccgattcagtgaagggt

SEQ ID NO: 128 - DNA, 24 bp, synthetic construct
  misc_feature 1..24: note = CDRH3
  source 1..24: mol_type = other DNA
gcacagggtacttctgcagatttc

SEQ ID NO: 129 - DNA, 15 bp, synthetic construct
  misc_feature 1..15: note = CDRH1
  source 1..15: mol_type = other DNA
tcttatgcaatgtct

SEQ ID NO: 130 - DNA, 51 bp, synthetic construct
  misc_feature 1..51: note = CDRH2
  source 1..51: mol_type = other DNA
gcaatccgttcttctggtggtactacttactatgccgattcagtgaagggt

SEQ ID NO: 131 - DNA, 24 bp, synthetic construct
  misc_feature 1..24: note = CDRH3
  source 1..24: mol_type = other DNA
tctcagtctactgttgcagattac

SEQ ID NO: 132 - DNA, 15 bp, synthetic construct
  misc_feature 1..15: note = CDRH1
  source 1..15: mol_type = other DNA
tcttatgcaatgcac

SEQ ID NO: 133 - DNA, 51 bp, synthetic construct
  misc_feature 1..51: note = CDRH2
  source 1..51: mol_type = other DNA
gcaatctcttcttctggtggtactaaatactatgccgattcagtgaagggt

SEQ ID NO: 134 - DNA, 33 bp, synthetic construct
  misc_feature 1..33: note = CDRH3
  source 1..33: mol_type = other DNA
ggtctgcagggtggtgcaggtgcatttgatatc

SEQ ID NO: 135 - DNA, 15 bp, synthetic construct
  misc_feature 1..15: note = CDRH1
  source 1..15: mol_type = other DNA
tcttatgcaatgtct

SEQ ID NO: 136 - DNA, 51 bp, synthetic construct
  misc_feature 1..51: note = CDRH2
  source 1..51: mol_type = other DNA
tctatctcttcttcttctggtactactcactatgccgattcagtgaagggt

SEQ ID NO: 137 - DNA, 18 bp, synthetic construct
  misc_feature 1..18: note = CDRH3
  source 1..18: mol_type = other DNA
ggtggtgcattcgattac

SEQ ID NO: 138 - DNA, 15 bp, synthetic construct
  misc_feature 1..15: note = CDRH1
  source 1..15: mol_type = other DNA
tcttatggtatgcac

SEQ ID NO: 139 - DNA, 51 bp, synthetic construct
  misc_feature 1..51: note = CDRH2
  source 1..51: mol_type = other DNA
ggtatctcttcttctggtggttctaaatactatgccgattcagtgaagggt

SEQ ID NO: 140 - DNA, 24 bp, synthetic construct
  misc_feature 1..24: note = CDRH3
  source 1..24: mol_type = other DNA
catggtcatcagcagatggatttc

SEQ ID NO: 141 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccagtctatctctcgttggctgaactggtatcagcagaaaccgggcaaggcgccaaaattgctgatttacgcaacttccactctgcagtctggtgtaccgtcccgtttctctggcagcggttctggtacggattttaccctgaccatctcaagcctccagcctgaagattttgccacctattattgtcagcaatcttactcttttccgtggacgttcgggcagggaactaaagtggaaattaaa

SEQ ID NO: 142 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccaggatatctctaattacctgaactggtatcagcagaaaccgggcaaggcgccaaaattgctgatttacgcagcatcctctctgcagtctggtgtaccgtcccgtttctctggcagcggttctggtacggattttaccctgaccatctcaagcctccagcctgaagattttgccacctattattgtcagcaatcttactcttttccgtacacgttcgggcagggaactaaagtggaaattaaa

SEQ ID NO: 143 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccagtctatctctcgttacctgaactggtatcagcagaaaccgggcaaggcgccaaaattgctgatttacgcagcatcctctctgcagtctggtgtaccgtcccgtttctctggcagcggttctggtacggattttaccctgaccatctcaagcctccagcctgaagattttgccacctattattgtcagcaatcttcttctactccgtggacgttcgggcagggaactaaagtggaaattaaa

SEQ ID NO: 144 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccaggatatctcttcttggctgaactggtatcagcagaaaccgggcaaggcgccaaaattgctgatttacgcagcatcccgtctgcagtctggtgtaccgtcccgtttctctggcagcggttctggtacggattttaccctgaccatctcaagcctccagcctgaagattttgccacctattattgtcagcaagcatactcttttccgtggacgttcgggcagggaactaaagtggaaattaaa

SEQ ID NO: 145 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccagtctatctctaattggctgaactggtatcagcagaaaccgggcaaggcgccaaaattgctgatttacgcaacttcccgtctgcagtctggtgtaccgtcccgtttctctggcagcggttctggtacggattttaccctgaccatctcaagcctccagcctgaagattttgccacctattattgtcagcaatcttactcttttccgtacacgttcgggcagggaactaaagtggaaattaaa

SEQ ID NO: 146 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccagtctatctctcgttggctgaactggtatcagcagaaaccgggcaaggcgccaaaattgctgatttacgcagcatcccgtctgcagtctggtgtaccgtcccgtttctctggcagcggttctggtacggattttaccctgaccatctcaagcctccagcctgaagattttgccacctattattgtcagcaatcttactctactccgtggacgttcgggcagggaactaaagtggaaattaaa

SEQ ID NO: 147 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccagtctatctctaattggctgaactggtatcagcagaaaccgggcaaggcgccaaaattgctgatttacgcaacttcccgtctgcagtctggtgtaccgtcccgtttctctggcagcggttctggtacggattttaccctgaccatctcaagcctccagcctgaagattttgccacctattattgtcagcaatcttactcttttccgctgacgttcgggcagggaactaaagtggaaattaaa

SEQ ID NO: 148 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccagtctatctctaattacctgaactggtatcagcagaaaccgggcaaggcgccaaaattgctgatttacgcaacttccactctgcactctggtgtaccgtcccgtttctctggcagcggttctggtacggattttaccctgaccatctcaagcctccagcctgaagattttgccacctattattgtcagcaatcttactcttttccgtggacgttcgggcagggaactaaagtggaaattaaa

SEQ ID NO: 149 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccaggatatctctcgttggctgaactggtatcagcagaaaccgggcaaggcgccaaaattgctgatttacgcagcatccactctgcagtctggtgtaccgtcccgtttctctggcagcggttctggtacggattttaccctgaccatctcaagcctccagcctgaagattttgccacctattattgtcagcaatcttactcttttccgctgacgttcgggcagggaactaaagtggaaattaaa

SEQ ID NO: 150 - DNA, 321 bp, synthetic construct
  misc_feature 1..321: note = VL
  source 1..321: mol_type = other DNA
gacattcaaatgacgcagagtccctcctcactgagtgctagcgtgggcgatcgtgtgacaattacttgtcgcgctagccagtctatctcttcttggctgaactggtatcagcagaaaccgggcaaggcgccaaaattgctgatttacgcagcatcccgtctgcagtctggtgtaccgtcccgtttctctggcagcggttctggtacggattttaccctgaccatctcaagcctccagcctgaagattttgccacctattattgtcagcaaacttactcttttccgtggacgttcgggcagggaactaaagtggaaattaaa

SEQ ID NO: 151 - DNA, 351 bp, synthetic construct
  misc_feature 1..351: note = VH
  source 1..351: mol_type = other DNA
gaagtacagttggtcgaaagtggcggtggcctcgtgcaaccgggtggttcactgcgtctgagctgcgccgcctcgggttttactttctcttcttatggtatgtcttgggttcgtcaggcgccgggcaagggtctcgaatgggtttcagcaatcaaaggttctggtggttctacttactatgccgattcagtgaagggtcgctttaccatttcccgtgacaactctaagaatactctgtatctgcagatgaactcgctgcgtgccgaagacacggccgtctattattgcgccaaaacttacggtggtgaaatcgattactggggtcagggcactttagtgaccgtctcatcg

SEQ ID NO: 152 - DNA, 351 bp, synthetic construct
  misc_feature 1..351: note = VH
  source 1..351: mol_type = other DNA
gaagtacagttggtcgaaagtggcggtggcctcgtgcaaccgggtggttcactgcgtctgagctgcgccgcctcgggttttactttctctgattatgcaatgtcttgggttcgtcaggcgccgggcaagggtctcgaatgggtttcaggtatctcttcttctggtggtactatctactatgccgattcagtgaagggtcgctttaccatttcccgtgacaactctaagaatactctgtatctgcagatgaactcgctgcgtgccgaagacacggccgtctattattgcgccaaagcaatcggttcttgggcagatatctggggtcagggcactttagtgaccgtctcatcg

SEQ ID NO: 153 - DNA, 351 bp, synthetic construct
  misc_feature 1..351: note = VH
  source 1..351: mol_type = other DNA
gaagtacagttggtcgaaagtggcggtggcctcgtgcaaccgggtggttcactgcgtctgagctgcgccgcctcgggttttactttctcttcttatgcaatgcactgggttcgtcaggcgccgggcaagggtctcgaatgggtttcagcaatctcttcttcttctggtactatctactatgccgattcagtgaagggtcgctttaccatttcccgtgacaactctaagaatactctgtatctgcagatgaactcgctgcgtgccgaagacacggccgtctattattgcgccaaagatttcggtgcagttttcgattactggggtcagggcactttagtgaccgtctcatcg

SEQ ID NO: 154 - DNA, 351 bp, synthetic construct
  misc_feature 1..351: note = VH
  source 1..351: mol_type = other DNA
gaagtacagttggtcgaaagtggcggtggcctcgcgcaaccgggtggttcactgcgtctgagctgcgccgcctcgggttttactttctcttcttatgcaatgcactgggttcgtcaggcgccgggcaagggtctcgaatgggtttcatctatcaaatcttctggtggttctaaaaattatgccgattcagtgaagggtcgctttaccatttcccgtgacaactctaagaatactctgtatctgcagatgaactcgctgcgtgccgaagacacggccgtctattattgcgccaaaggttacggtgcatctttcgatttctggggtcagggcactttagtgaccgtctcatcg

SEQ ID NO: 155 - DNA, 348 bp, synthetic construct
  misc_feature 1..348: note = VH
  source 1..348: mol_type = other DNA
gaagtacagttggtcgaaagtggcggtggcctcgtgcaaccgggtggttcactgcgtctgagctgcgccgcctcgggttttactttctcttcttatgcaatgcactgggttcgtcaggcgccgggcaagggtctcgaatgggtttcagcaatctcttcttctggtggttctatctactatgccgattcagtgaagggtcgctttaccatttcccgtgacaactctaagaatactctgtatctgcagatgaactcgctgcgtgccgaagacacggccgtctattattgcgccaaacagggtctgtctttcgatatctggggtcagggcactttagtgaccgtctcatcg

SEQ ID NO: 156 - DNA, 351 bp, synthetic construct
  misc_feature 1..351: note = VH
  source 1..351: mol_type = other DNA
gaagtacagttggtcgaaagtggcggtggcctcgtgcaaccgggtggttcactgcgtctgagctgcgccgcctcgggttttactttctcttcttatgcaatgtcttgggttcgtcaggcgccgggcaagggtctcgaatgggtttcagcaatctctggttcttctggttctacttactatgccgattcagtgaagggtcgctttaccatttcccgtgacaactctaagaatactctgtatctgcagatgaactcgctgcgtgccgaagacacggccgtctattattgcgccaaagcacagggtacttctgcagatttctggggtcagggcactttagtgaccgtctcatcg

SEQ ID NO: 157 - DNA, 351 bp, synthetic construct
  misc_feature 1..351: note = VH
  source 1..351: mol_type = other DNA
gaagtacagttggtcgaaagtggcggtggcctcgtgcaaccgggtggttcactgcgtctgagctgcgccgcctcgggttttactttctcttcttatgcaatgtcttgggttcgtcaggcgccgggcaagggtctcgaatgggtttcagcaatccgttcttctggtggtactacttactatgccgattcagtgaagggtcgctttaccatttcccgtgacaactctaagaatactctgtatctgcagatgaactcgctgcgtgccgaagacacggccgtctattattgcgccaaatctcagtctactgttgcagattactggggtcagggcactttagtgaccgtctcatcg

SEQ ID NO: 158 - DNA, 360 bp, synthetic construct
  misc_feature 1..360: note = VH
  source 1..360: mol_type = other DNA
gaagtacagttggtcgaaagtggcggtggcctcgtgcaaccgggtggttcactgcgtctgagctgcgccgcctcgggttttactttctcttcttatgcaatgcactgggttcgtcaggcgccgggcaagggtctcgaatgggtttcagcaatctcttcttctggtggtactaaatactatgccgattcagtgaagggtcgctttaccatttcccgtgacaactctaagaatactctgtatctgcagatgaactcgctgcgtgccgaagacacggccgtctattattgcgccaaaggtctgcagggtggtgcaggtgcatttgatatctggggtcagggtactctggttactgtatcatcg

SEQ ID NO: 159 - DNA, 345 bp, synthetic construct
  misc_feature 1..345: note = VH
  source 1..345: mol_type = other DNA
gaagtacagttggtcgaaagtggcggtggcctcgtgcaaccgggtggttcactgcgtctgagctgcgccgcctcgggttttactttctcttcttatgcaatgtcttgggttcgtcaggcgccgggcaagggtctcgaatgggtttcatctatctcttcttcttctggtactactcactatgccgattcagtgaagggtcgctttaccatttcccgtgacaactctaagaatactctgtatctgcagatgaactcgctgcgtgccgaagacacggccgtctattattgcgccaaaggtggtgcattcgattactggggtcagggcactttagtgaccgtctcatcg

SEQ ID NO: 160 - DNA, 351 bp, synthetic construct
  misc_feature 1..351: note = VH
  source 1..351: mol_type = other DNA
gaagtacagttggtcgaaagtggcggtggcctcgtgcaaccgggtggttcactgcgtctgagctgcgccgcctcgggttttactttctcttcttatggtatgcactgggttcgtcaggcgccgggcaagggtctcgaatgggtttcaggtatctcttcttctggtggttctaaatactatgccgattcagtgaagggtcgctttaccatttcccgtgacaactctaagaatactctgtatctgcagatgaactcgctgcgtgccgaagacacggccgtctattattgcgccaaacatggtcatcagcagatggatttctggggtcagggcactttagtgaccgtctcatcg